(19) United States
(12) Patent Application Publication
(10) Pub. No.: US 2025/0125010 A1
(43) Pub. Date: Apr. 17, 2025

(54) DETERMINING FRAGMENTOMIC SIGNATURES BASED ON LATENT VARIABLES OF NUCLEIC ACID MOLECULES

(71) Applicant: Personalis, Inc., Fremont, CA (US)

(72) Inventors: Zeid M. RUSAN, Fremont, CA (US); Nicholas A. PHILLIPS, Palo Alto, CA (US); Jason HARRIS, Belmont, CA (US); Ning ZHANG, Fremont, CA (US)

(21) Appl. No.: 18/704,272

(22) PCT Filed: Oct. 31, 2022
(86) PCT No.: PCT/US2022/078956

§ 371 Date: Apr. 24, 2024

Related U.S. Application Data
(60) Provisional application No. 63/274,330, filed on Nov. 1, 2021.

(51) Int. Cl. G16B 30/10; G16B 20/20; G16B 40/30

(52) U.S. Cl. CPC G16B 30/10; G16B 20/20; G16B 40/30

(57) ABSTRACT

A method of predicting a classification of a disease of a subject based on fragmentomic signatures can include accessing sequence data of a biological sample of a subject. The method can also include generating, based on the sequence data, a set of sequence-size values. Each sequence-size value of the set can correspond to a size of a sequence of the sequence data. The method can also include determining fragmentomic signature amplitudes of the subject by projecting the set of sequence-size values onto latent variables of a fragmentomic signature. The latent variables can be generated by applying one or more signal-separation algorithms to other sequence-size values obtained from one or more reference biological samples. The method can also include generating a result by processing the fragmentomic signature amplitudes using a machine-learning model. The result can include a classification predictive of whether the subject has a particular disease.

### CROSS-REFERENCES TO RELATED APPLICATIONS

This application claims priority to U.S. Provisional Patent Application No. 63/274,330, entitled “Determining Gene Signatures Based On Latent Variables Of Nucleic Acid Molecules,” filed on Nov. 1, 2021, the contents of which are hereby incorporated by reference in their entirety for all purposes.

### BACKGROUND

Next generation sequencing can be used to identify genetic characteristics of subjects. For example, whole genome sequencing can be used to reveal somatic variants in sequence data of a subject, some of which corresponding to tumor DNA. In addition, recent research in cell-free DNA fragmentomic features such as fragment length and end-motif has expanded the potential for accurately diagnosing subjects using plasma samples. For example, such research has facilitated the discovery of fragment-length features that correspond to different types of tumors. There also have been efforts to develop the above techniques to detect cancer in subjects.

Despite such efforts, accurately detecting cancer from plasma samples has remained elusive. A contributing factor for such difficulty is that quantity of tumor DNA in the plasma samples can vary substantially, depending on the tumor type, the stage of disease progression, and the degree of tumor cell DNA release having access to the circulatory system (tumor “shedding”). Due to these varying characteristics, identifying particular features of the tumor DNA (e.g., fragment length, end motif) has been challenging. In addition, tumor DNA concentration in plasma samples can be low, to the extent that an accurate diagnosis for the subject becomes difficult.

### SUMMARY

In some embodiments, a method of predicting a classification of a disease of a subject based on fragmentomic signatures based on size distribution of nucleic acid molecules is provided. The method can include accessing sequence data of a biological sample of a subject. The method can also include generating, based on the sequence data, a set of sequence-size values. Each sequence-size value of the set can correspond to a size of a sequence of the sequence data. The method can also include determining fragmentomic signature amplitudes of the subject by projecting the set of sequence-size values onto latent variables of a fragmentomic signature. The latent variables can be generated by applying one or more signal-separation algorithms to other sequence-size values obtained from one or more reference biological samples. The method can generate a result by processing the fragmentomic signature amplitudes using a machine-learning model. The result can include a classification predictive of whether the subject has a particular disease. The method can include outputting the result.

In some embodiments, a method of predicting a classification of a disease of a subject based on fragmentomic signatures based on distribution of end-motif frequencies is provided. The method can include accessing sequence data of a biological sample of a subject. The method can also include generating, based on the sequence data, a set of end-motif sequence data. Each end-motif sequence data of the set identifies a number or relative frequency of nucleic acid molecules having an ending sequence that correspond to a particular end-motif. The method can also include determining fragmentomic signature amplitudes of the subject by projecting the set of end-motif sequence data onto latent variables of a fragmentomic signature. The latent variables can be generated by applying one or more signal-separation algorithms to other end-motif sequence data obtained from one or more reference biological samples. The method can generate a result by processing the fragmentomic signature amplitudes using a machine-learning model. The result can include a classification predictive of whether the subject has a particular disease. The method can include outputting the result.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by some embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures.

FIG. 1 shows a schematic diagram that illustrates the process of generating latent variables from sequence data and using the latent variables to predict presence of cancer, according to some embodiments.

FIG. 2 includes a flowchart illustrating an example of a method of for predicting a classification of a disease of a subject based on fragmentomic signatures determined based on size distributions of nucleic acid molecules, according to some embodiments.

FIG. 3 shows a schematic diagram that illustrates an example technique for generating sequence-size values, according to some embodiments.

FIG. 4 shows an example diagram that illustrates a process for using signal-separation algorithms to generate a set of unmixed images, according to some embodiments.

FIG. 5 shows a schematic diagram that illustrates an example technique for generating a set of sequence-size latent variables, according to some embodiments.

FIG. 6 shows a schematic diagram that illustrates an example technique of using independent component analysis algorithm on whole exome sequence data for generating fragmentomic signatures, according to some embodiments.

FIG. 7 shows a set of latent variables generated by applying a signal-separation algorithm on sequence-size values corresponding to a biological sample of a normal subject.

FIG. 8 shows a set of latent variables generated by applying a signal-separation algorithm on sequence-size values corresponding to a biological sample of another normal subject.

FIG. 9 shows a set of latent variables generated by applying a signal-separation algorithm on sequence-size values corresponding to a biological sample of a subject diagnosed with colorectal cancer.

FIG. 10 shows a set of latent variables generated by applying a signal-separation algorithm on sequence-size values corresponding to a biological sample of another subject diagnosed with colorectal cancer.

FIG. 11 shows a schematic diagram that illustrates a technique for predicting enrichment of DNA molecules originating from loci with different epigenetic states based on fragmentomic signatures, according to some embodiments.

FIG. 12 shows a schematic diagram that illustrates a process for pre-processing raw sequence-size distributions by projecting them onto latent variables, transforming the distributions into a set of fragmentomic signature amplitudes, and using the amplitudes to detect cancer-related gene mutations, according to some embodiments.

FIG. 13 shows a schematic diagram that illustrates an example technique of using independent component analysis algorithm on hybridization capture samples to generate a set of latent variables, according to some embodiments.

FIG. 14 shows a schematic diagram that illustrates example techniques for monitoring cancer relapse using hand-crafted features or latent variable features, according to some embodiments.

FIG. 15 shows a set of receiver operating characteristic (ROC) curves that show accuracy levels of using fragmentomic signatures for classification of diseases.

FIG. 16 includes a flowchart illustrating an example of a method for predicting a classification of a disease of a subject based on fragmentomic signatures determined based on end-motif frequencies of nucleic acid molecules, according to some embodiments.

FIG. 17 illustrates an example of a computer system for implementing some of some embodiments disclosed herein.

### DETAILED DESCRIPTION

Fragmentomics generally refer to an analysis of fragmentation patterns of cell-free DNA, including fragment size and end-motif. These fragmentation patterns can be associated with epigenetic signatures specific to tissue type and cancer. It is believed that, in part, differences in normal and cancer cell histone and associated regulatory proteins governing chromatin architecture, and the interrelated gene transcriptional landscape, can be manifested as DNA-fragment length distribution differences. Despite these advances, it is difficult to use such features in developing effective therapies (e.g., neoantigen vaccines). For example, tumor DNA levels are usually low in plasma samples, and such limited data may not allow results to reach sufficient accuracy in predicting whether a subject has cancer. In another example, somatic variants of non-tumor origin found in cell-free DNA, including those of clonal hematopoiesis, can complicate variant calling and detection of tumor DNA. In yet another example, the availability of tumor sequence data of the sequence knowledge databases can be inconsistent, such that they cannot adequately serve as training data for training machine-learning models for predicting whether a subject has cancer.

To address these challenges, the present techniques can include predicting a classification of a disease for a subject based on a fragmentomic signature. As used herein, the fragmentomic signature refers to one or more signatures of size and/or end-motif distributions of nucleic acid molecules that can be predictive of the classification of the disease. For example, the fragmentomic signature can represent estimated distributions of fragment lengths (interchangeably referred to as “sizes”) of sequences that align to each genomic region of a set of genomic regions that can be predictive of the classification of the disease. Additionally or alternatively, the fragmentomic signature can represent estimated distributions of X-bp 5′ and 3′ sequence identities or “end-motifs” (e.g., CCCA) that can be predictive of the classification of the disease.

The fragmentomic signature can include one or more latent variables generated based on applying a blind-source separation (BSS) algorithm to size and/or end-motif distributions of nucleic acid molecules obtained from a reference cohort. Each latent variable can identify an estimated size and/or end-motif distribution of sequences that is variably enriched across the set of genomic regions and defines a new basis vector for sequence-size and/or end-motif data. In some instances, the one or more latent variables are determined based on sequence data obtained from reference samples with disease diagnoses. In some instances, the one or more latent variables identify an estimated distribution of sequence reads that have ending sequences corresponding to a particular set of end motif. The fragmentomic signature may then be used to predict whether the subject has cancer (for example).

The techniques for predicting a classification of a disease for a subject based on the fragmentomic signature can be initiated by accessing sequence data of a biological sample of a subject. In some instances, the sequencing data identifies a plurality of nucleic-acid sequences, which were obtained by sequencing a plurality of cell-free DNA molecules of the biological sample. The plurality of cell-free DNA molecules can include circulating-tumor DNA molecules. Additionally, or alternatively, the sequence data can also identify sequence reads corresponding to a plurality of somatic variants detected from the biological sample. The plurality of somatic variants can be detected by aligning each sequence read of the sequence data to a reference sequence (e.g., a human reference genome).

In some instances, the reference sequence includes “normal” or “healthy” sequences obtained from healthy blood cells (e.g., leukocytes), buccal cells, and/or root hair root cells of one or more subjects. The cells can be identified as healthy in a variety of ways, e.g., when a person is previously diagnosed to not have a specific type of cancer or the sample can be obtained from tissue that is not likely to contain cancerous or premalignant cells. In some instances, the normal sequences are obtained by: (i) separating, from a blood sample, the plasma from the buffy coat that includes leukocytes and peripheral blood mononuclear cells; (ii) isolating the DNA from the buffy coat; and (iii) determining the normal sequences from the isolated DNA. Example techniques for determining normal sequences from a biological sample are further described in U.S. Pat. No. 10,125,399, the contents of which being incorporated herein by reference in its entirety for all purposes.

Based on the sequence data, a set of sequence-size values (e.g., a two-dimensional matrix of sequence-size values) can be generated. For example, the set of sequence-size values can include a two-dimensional matrix of sequence-size values, in which a first dimension defining a sequence size (e.g., 30 bp) is associated with a second dimension that identifies a number of sequences that correspond to the sequence size (e.g., 50 counts). The set of sequence-size values can include, for each nucleic-acid sequence of the sequence data that aligns to a corresponding genomic region of a set of genomic regions, a sequence-size value that represents a size of the sequence. Each sequence represented by a corresponding sequence-size value can include a DNA fragment within a certain size (bp) range (e.g., size range between 60 bp and 600 bp). In some instances, the set of genomic regions are identified using a reference sequence (e.g., a human reference genome). Additionally, or alternatively, the set of sequence-size values can be transformed into a set of empirical probability mass functions, in which each empirical probability function can be generated based on sequence-size values of the nucleic-acid sequences that align to the corresponding genomic region. In some instances, size-distribution data representing the set of sequence-size values is determined from the sequence data of the subject. For example, the set of sequence-size values can be transformed into an empirical probability mass function (PMF). As used herein, the PMF refers to a function that estimates the probability that a discrete random variable is exactly equal to some value. The set of PMFs can be used as input to determine the set of latent variables.

To predict the classification of the disease using the set of sequence-size values of the biological sample, the fragmentomic signature can be determined. To determine the fragmentomic signature, a set of latent variables can first be generated. As used herein, the term “latent variable” refers to an estimated distribution of sequence sizes—a pattern encoded as a numeric vector of sequence-size signed weights—corresponding to one of several latent sources of variation underlying the sequence data that align to the set of genomic regions. The set of latent variables can be determined by applying one or more signal-separation algorithms to sequence-size values obtained from one or more reference samples. In some instances, the reference samples include biological samples obtained from other subjects with disease diagnoses (e.g., cancer, healthy). Additionally or alternatively, to monitor progression of disease, the reference samples can include biological samples obtained from the same subject but at different time points. For example, the reference sample can correspond to a biological sample of the subject that was obtained 2 years prior to the time the current biological sample is obtained.

The one or more signal-separation algorithms can include a blind-source separation algorithm, an independent component analysis algorithm and/or a non-negative matrix factorization algorithm. As used herein, the blind-source separation algorithm can be a technique for separating a set of source signals (e.g., latent variables) from a set of mixed signals (e.g., sequence-size values), without the aid of information (or with very little information) about the source signals or the mixing process. Additionally, or alternatively, the one or more signal-separation algorithms can be one or more other unsupervised machine-learning techniques. In some instances, a subset of latent variables (alternatively referred to as a “fixed” latent variables) are selected by applying a clustering algorithm to the set of latent variables and identifying centroids or medoids of each cluster generated by the clustering algorithm. The fixed latent variables can be used as the fragmentomic signature, which can represent nucleic acid molecules produced by different biological processes reflected in the one or more reference samples.

The set of sequence-size values of the biological sample can then be projected onto the latent variables of the fragmentomic signature. For example, the size-distribution data determined from the set of sequence-size values can be projected onto the fixed latent variables to generate one or more latent variable coefficients (interchangeably referred to as “amplitudes”). The fragmentomic-signature amplitudes can represent the enrichment of different groups of nucleic acid molecules in the biological sample.

A result can be generated by processing the fragmentomic-signature amplitudes using a machine-learning model, in which the result includes a classification predictive of whether the subject has a particular disease. Additionally, or alternatively, the result includes a classification predictive of whether the subject has a particular type of cancer, and the result can be used to predict a treatment for the subject and/or predict how often the treatment should be administered to the subject.

In some instances, the fragmentomic signature can be used to generate a result for performing other types of tasks. An example task can include measuring, based on the fragmentomic signature, enrichment of protein molecule sets linked to epigenetic states in sequence-size data based on identifying certain latent variables as the signals carried by independently regulated molecule sets, according to some embodiments. For example, enrichment of the biological sample for nucleic acid molecules known to be associated with chromatosomes of a particular genomic region or a specific allele can be used to predict binding of chromatosomes and enrichment of silenced heterochromatin states at the corresponding cellular loci contributing to the sequencing data. In another example, enrichment of the biological sample for nucleic acid molecules associated with cancer in the sequence data can be used to predict whether a particular allele came from cancer cells. In yet another example, enrichment of the biological sample for nucleic acid molecules associated with gene expression in the sequence data can be used to predict whether a particular allele contributing to the sequencing data is being expressed.

Yet another task includes estimating the size variability and central tendency of the nucleic acid molecules of the biological sample known to be bound to the protein molecules such as nucleosomes and transcription factors. Another task includes inferring the spacing and linker DNA size of different proteins by comparing size differences between peaks in one or more multimodal latent variables. Other tasks include predicting the relative abundance of short and long sequence-size species of one or more multimodal latent variables, and, inferring cell-free DNA degradation kinetics of the fragmentomic signature from different rates of change in latent variable space.

Additionally or alternatively, the fragmentomic signature can be used for data denoising, a function that can be advantageous especially in instances of low data amount. Projecting raw data (e.g., the size-distribution data) onto latent variable space reduces downstream overfitting of models and improves the accuracy of statistical tests by virtue of dropping noise or other data deemed uninteresting. Yet another example can include the use of latent variables for dimensionality reduction by projecting raw data onto a few latent variables of interest to generate a few numeric amplitudes (e.g., two, amicable for depicting data in 2D plots). The numeric amplitudes between the wild-type allele supporting reads and mutant allele supporting reads can be compared, such that false positive mutations caused by PCR or sequencing errors can be filtered out.

Accordingly, some embodiments of the present disclosure provide a technical advantage over conventional techniques by using signal-separation and clustering algorithms to generate a fragmentomic signature to represent an accurate and generalizable model of independent sources of variation underlying sequence-size and/or end-motif data in a biological sample. For example, blind source separation algorithms can identify hidden but independent factors from sequence data that can be used to predict a disease or a condition of the subject. In some instances, the fragmentomic signature can be used to predict characteristics of different nucleic acid or protein molecules, which can be used for diagnosis or prognosis of different epigenetic states). As described above, the fragmentomic may identify inherent properties of nucleic acid molecules that are useful for interpreting disease phenotypes by virtue of understanding what they represent.

All of the aforementioned technical advantages lead to downstream benefits to computational efficiency and to supervised machine-learning model interpretation, generalizability and performance. The fragmentomic signature that includes the fixed latent variables can then be used to predict whether the biological sample includes tumor DNA and/or can also be used to predict a particular stage of cancer. Further, the use of the fragmentomic signature allows accurate prediction of whether the subject is afflicted with a particular disease, even when a detected amount of tumor DNA is low. Therefore, the present techniques facilitate accurate and reliable detection of diseases in subjects by applying the machine-learning models to sequence data derived from cell-free plasma samples.

The following examples are provided to introduce certain embodiments. In the following description, for the purposes of explanation, specific details are set forth in order to provide a thorough understanding of examples of the disclosure. However, it will be apparent that various examples may be practiced without these specific details. For example, devices, systems, structures, assemblies, methods, and other components may be shown as components in block diagram form in order not to obscure the examples in unnecessary detail. In other instances, well-known devices, processes, systems, structures, and techniques may be shown without necessary detail in order to avoid obscuring the examples. The figures and description are not intended to be restrictive. The terms and expressions that have been employed in this disclosure are used as terms of description and not of limitation, and there is no intention in the use of such terms and expressions of excluding any equivalents of the features shown and described or portions thereof. The word “example” is used herein to mean “serving as an example, instance, or illustration.” Any embodiment or design described herein as an “example” is not necessarily to be construed as preferred or advantageous over other embodiments or designs.

### I. Overview of Predicting a Classification of a Disease of a Subject Based on Fragmentomic Signatures

Paired tumor biopsy and cell-free DNA plasma studies in subjects have revealed differing characteristics between the two corresponding somatic variant call sets. Differences between paired tumor and plasma somatic variant call sets were found to be highly variable, in that the differences can be affected based on tumor type, stage, heterogeneity, and timing of acquisition of each sample.

Global patterns of tumor DNA fragment lengths have been shown to be shorter compared to those of normal cell-free DNA. By enriching biological samples for known tumor variants then examining fragment lengths inferred from sequence reads generated from normal and enriched biological sample pairs, techniques for predicting disease-status differentiating features can be implemented. Nonetheless, it can be challenging to distinguish tumor DNA from normal DNA solely based on their size distributions. Further, an amount of tumor DNA in a biological sample can vary, in which previous techniques cannot accurately predict whether a particular set of sequences are from tumor DNA. Due to this difficulty, incorporating DNA fragment lengths of tumor DNA into various techniques (e.g., somatic variant calling) has remained elusive.

To address these challenges, the present techniques can include determining fragmentomic signature based on latent variables generated by a BSS algorithm. The fragmentomic signature can be used to predict a classification of a disease of a subject. The latent variables can be generated by applying one or more machine-learning models (e.g., one or more unsupervised machine-learning models, BSS algorithms) to a plurality of sets of sequence-size values obtained from reference samples. A reference sample can be obtained from a subject with disease diagnosis (e.g., cancer) and/or a healthy subject. A size of each sequence can be measured to identify a sequence-size value. Each set of the plurality of sets of sequences-size values can be associated with a genomic region of a set of genomic regions identified in a reference genome. The plurality of sets of sequence-size values can be represented as a matrix or any data structure that represents size distribution of the sequence-size values. Additionally or alternatively, each set of sequence-size values can be transformed into an empirical probability mass function (PMF), such that the plurality of sets of sequence-size values can be represented by a set of PMFs. Each latent variable represents a set of weighted fragment sizes associated with an independent biological process, technical artifact, or noise source. The biologically meaningful latent variables (e.g., fixed latent variables) can be selected and used as fragmentomic signatures. In some instances, a clustering algorithm is applied to the latent variables to select the fixed latent variables that form the fragmentomic signature.

Fragmentomic signature amplitudes of a particular subject (e.g., a subject with without disease diagnosis) can be determined by projecting the size distribution of sequences of a biological sample of the particular subject onto the set of biologically meaningful latent variables. In some instances, the fragmentomic signature amplitudes can be used to predict whether the sequence data of the subject include somatic variants of known tumor origin, which can facilitate cell-free variant calling and early cancer detection. In some instances, the fragmentomic signature amplitudes are processed by a classifier model to predict whether a person has certain disease (e.g., cancer).

### A. Example Technique of Using Fragmentomic Signatures to Predict Disease in Subjects

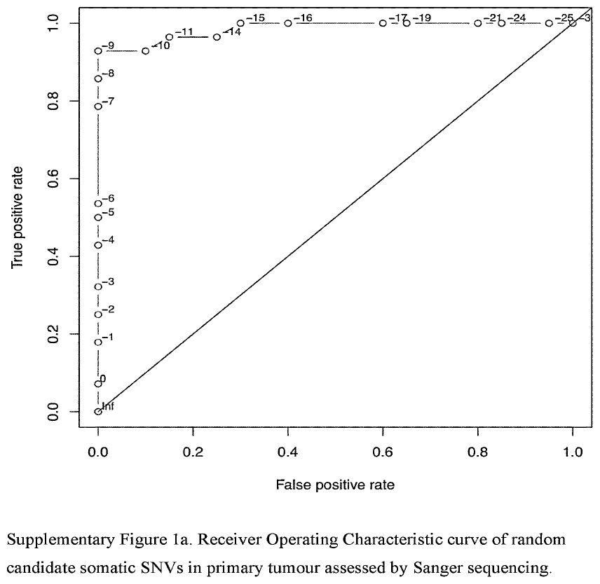

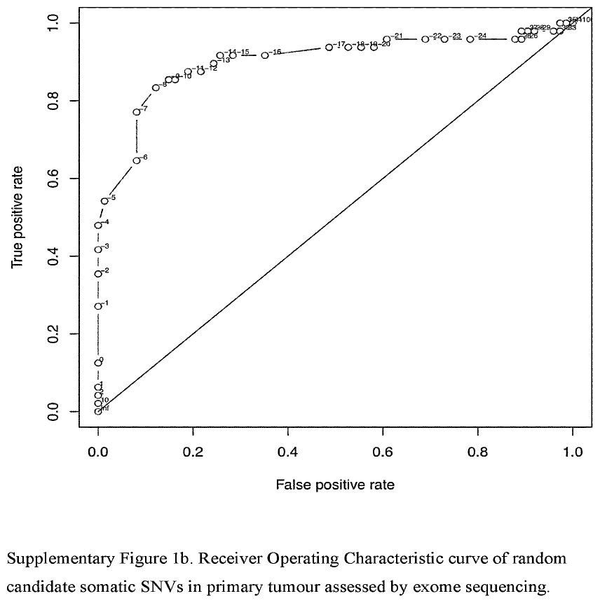

FIG. 1 shows a schematic diagram 100 that illustrates the process of generating latent variables from sequence data and using the latent variables to predict presence of cancer, according to some embodiments. The process 100 includes: (i) a first stage 102 for generating the set of latent variables using blind-source separation algorithm on reference samples; and (ii) a second stage 104 for projecting sequence data of new samples onto the fixed latent variables.

i. First Stage

At step 106, cell-free DNA molecules (alternatively referred to as “cfDNA”) can be obtained from different reference samples. The cfDNA can be generated as a result of several biological processes such as apoptosis and necrosis. These cfDNA from different sources may have different fragment size distributions, which can be separated by BSS algorithms. In some instances, the reference samples include biological samples (e.g., serum or plasma samples) obtained from subjects with disease diagnoses (e.g., cancer). As a result, the latent variables determined for the above reference samples can be used as latent variables (or “fixed latent variables”) that are compared with size distributions of sequence data determined from another biological sample of another subject.

At step 108, size distributions of the cell-free DNA molecules can be determined. A set of sequence-size values can be identified for each genomic region of N genomic regions of a reference genome. Each sequence-size value of the set of sequence-size values can identify a size of a corresponding sequence read (e.g., paired-end read fragment insert size between DNA adapter sequences, fragment-length in bp) that aligned to the genomic region. Then, for each reference sample, a fragment-size distribution can be generated for the sets of sequence-size values. Only sequence-size values between 50 and 550 bp are used in the analysis. In some instances, a PMF is generated for each set of sequence-size values to determine the size distributions, in which each sequence-size value of the set is normalized by total number of sequences in the set.

At step 110, The size distributions of the sequence-size values can be processed by blind-source separation algorithms to generate a set of latent variables. In some instances, the set of PMFs are used as input data (e.g., a matrix that includes N×501 dimensions) for one or more blind-source separation algorithms. In this example, BSS algorithms included an independent component analysis (ICA) algorithm (e.g., fastICA) or a non-negative matrix factorization (NMF) algorithm. Depending on the type of the BSS algorithm, additional formatting of input data was performed. For example, X matrices inputted into non-negative matrix factorization algorithm were raw PMFs as described herein. In another example, X matrices inputted into the independent component analysis algorithms were first mean centered then scaled to unit variance. The BSS algorithms were executed multiple times until stochastic initial states achieved convergence (e.g., solution reached, minimization or maximization criteria met, maximized non-Gaussianity for ICA).

After BSS was performed on each sample, the latent variables from the J reference samples can be formed into conserved clusters using a clustering algorithm (e.g., K-means clustering algorithm). The centroid or medoid of each cluster can be selected, at which the centroid or medoid can be used as fixed latent variables that can represent cfDNA from different biological processes. Additionally or alternatively, a sequence-size PMF across the whole genome can be identified for each of the J reference samples. This J×501 (50-550 bp) matrix can be used as the input for one or more BSS algorithms. The output latent variables from the BSS algorithm can be directly used as fixed latent variables. The fixed latent variables can be collectively used as the fragmentomic signature to predict cancer in other biological samples.

ii. Second Stage

For the second stage 104, cell-free DNA molecules can be obtained from another biological sample. The other biological sample can be obtained from another subject with an unknown disease diagnosis. The other biological sample can be obtained from a subject for which a cancer treatment and surgery has been performed. In some instances, the other biological sample is obtained from the same subject from which the reference samples were obtained, but at a different time point of a time period. The other biological sample can be compared with the fixed latent variables of the fragmentomic signature to predict classification and/or monitor progress of cancer (for example) for the subject. At step 112, size-distribution data of cfDNA can be determined for the other subject. The size-distribution data can include one or more PMFs. The process for determining the size distribution is described in step 108 of FIG. 1.

At step 114, size-distribution data representing the sequence sizes of the other subject can be projected onto the fixed latent variables of the fragmentomic signature to generate fragmentomic signature amplitudes of the other subject. To calculate the fragmentomic signature amplitudes, PMF matrix representing the size-distribution data of the other subject are right-multiplied by the inverse of latent variables matrix. Depending on the BSS algorithm used for latent variables generation, additional formatting of input PMF such as scaling, whitening can be performed. Additionally or alternatively, similarity measurements (e.g., cosine similarity or Pearson correlation) can also be used to measure the enrichment of each fixed latent variable in the fragment size PMF. In some instances, the fragment size PMF for the other sample is generated from the size distribution of sequence sizes, at which the PMF can be compared to or projected onto the k fixed latent variables. In some instances, the 1×k feature amplitudes vector is selected to be used as a low dimension representation of the PMF for downstream analysis.

At step 116, downstream analysis can be performed to predict whether the other subject has cancer based on the fragmentomic signature amplitudes of the other subject. In some instances, a machine-learning model trained using the latent variables as features is configured to perform one or more predictive tasks including: (i) a prediction of whether the other subject carries cancer-related gene mutations; (ii) a prediction of whether the other subject has a disease (e.g., cancer); (iii) a prediction of whether the other subject has a particular type of disease (e.g., liver cancer); (iv) a prediction of a stage of the disease (e.g., stage IV cancer); and (v) a prediction of whether the other subject has recovered from the disease in response to a particular treatment.

In some embodiments, the machine-learning model includes more than one model (e.g., 1, 2, 3, 4, 5, 6, 7, 8, 9, or 10 machine-learning models). In some instances, the trained machine-learning model includes a deep neural network. Deep neural network can be used to capture the internal structure of increasingly larger and high-dimensional data sets (e.g., nucleic acid sequence data). Deep neural networks can identify high-level features, improve performance over traditional statistical models, increase interpretability, and provide additional understanding about the structure of the nucleic acid sequence data.

Other types of machine-learning models can include one or more of gradient boosting decision trees (e.g., XGBoost framework, LightGBM framework), bagging procedures, boosting procedures, support vector machines, and/or random forest algorithms. For example, gradient boosting can correspond to a type of machine learning technique that can be used for regression and classification problems, and for producing a prediction model that may include an ensemble of weak prediction models, e.g., decision trees. In some instances, a gradient boosted decision tree can include, for example, an XGBoost framework or a LightGBM framework.

The machine-learning model may include hyperparameters. Hyperparameters can be a configuration that is external to the model and whose value are not be estimated from data (e.g., training data, input data). In some instances, hyperparameters are tuned, e.g., tuned to solve a given predictive modeling problem. In some instances, a hyperparameter is used to help estimate model parameters. The hyperparameters can be specified by a user. In some instances, a hyperparameter can be determined using a set of heuristic algorithms.

B. Methods for Predicting a Classification of a Disease of a Subject Based on Fragmentomic Signatures Determined from Size Distributions of Nucleic Acid Molecules

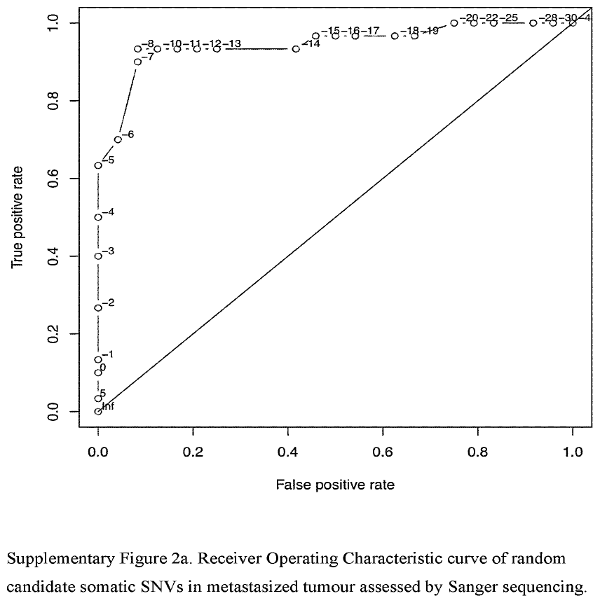

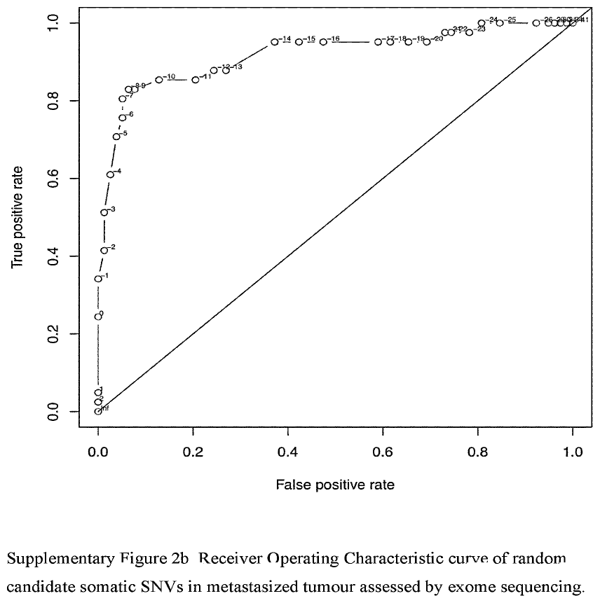

FIG. 2 includes a flowchart 200 illustrating an example of a method of for determining a fragmentomic signature of a subject based on size distributions of nucleic acid molecules of a biological sample, according to some embodiments. Some of the operations described in flowchart 200 may be performed by a computer system (e.g., a computer system 1700 of FIG. 17). Although flowchart 200 may describe the operations as a sequential process, in various embodiments, many of the operations may be performed in parallel or concurrently. In addition, the order of the operations may be rearranged. An operation may have additional steps not shown in the figure. Furthermore, some embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

At step 202, sequence data of a biological sample of a subject can be accessed. In some instances, the sequence data correspond to a plurality of cell-free DNA molecules of the biological sample, and the plurality of cell-free DNA molecules include circulating-tumor DNA molecules. The sequence data can also include sequences corresponding a plurality of somatic variants detected from the biological sample.

At step 204, based on the sequence data, a set of sequence-size values (e.g., a two-dimensional matrix of sequence-size values) can be generated. A sequence-size values can include, for each sequence of the sequence data that aligns to a corresponding genomic region of a set of genomic regions, a sequence-size value corresponding to a size of the sequence. Each sequence represented by a corresponding sequence-size value includes a DNA fragment having a size within certain range. In some instances, the set of genomic regions are identified using a reference sequence (e.g., a human reference genome). The set of sequence-size values can be an empirical probability mass function (PMF) generated based on the sequences of the sequence data that align to the corresponding genomic region. In some instances, a PMF is generated for the set of sequence-size values to determine the size distribution of the sequence data, in which each sequence-size value of the set is normalized by total number of sequences in the biological sample.

At step 206, the set of sequence-size values can be compared with or projected onto latent variables of a fragmentomic signature. The fragmentomic signature can include one or more signatures of size distributions of nucleic acid molecules that can be predictive of the classification of the disease. The fragmentomic signature can include one or more fixed latent variables. The one or more fixed latent variables can be determined by: (i) applying one or more signal-separation algorithms to sequence-size values obtained from one or more reference samples to generate a set of latent variables; and (ii) applying a clustering algorithm to select a subset of the set of latent variables, in which the subset correspond to the fixed latent variables. In some instances, the reference samples include biological samples (e.g., tissue, plasma sample) obtained from subjects with disease diagnoses (e.g., cancer). Additionally or alternatively, the reference samples can include a biological sample obtained from the same subject but at another time point. In some instances, each latent variable of the set of latent variables includes a histogram or weight vector that represents an estimated size distribution of the nucleic acid molecules produced by different biological processes. The one or more signal-separation algorithms can be a blind-source separation algorithm that can include an independent component analysis algorithm and/or a non-negative matrix factorization algorithm. In some instances, a clustering algorithm is applied to the latent variables generated from different reference samples to determine the subset of the set of latent variables. The subset of latent variables can include the centroids or medoids selected from the identified clusters of latent variables.

Additionally or alternatively, derivatives of latent variables can be determined by applying transformations such as scaling, translating, averaging, frequency domain converting (e.g., fast Fourier transforming) and other similar transformations.

In some instances, given that the BSS algorithms have the record of estimating signals carried by physically separate entities, the set of latent variables can be predictive of protein molecule sets (e.g., mono-nucleosome, di-nucleosome, mono-chromatosome, di-chromatosome and transcription factor complexes) that bind and protect cell-free DNA. In some instances, the latent variables can be predictive of potentially novel nucleic acid and/or protein entities and their corresponding structures based on their associated DNA fragmentomic signatures.

In addition, for latent variables corresponding to epigenetic states, density features such as variance and central tendency (e.g., peak sequence-size) can be predictive of the degree of intercellular heterogeneity of bound DNA and thus of the proteins enabling the epigenetic states. Also, the relative spacing and average linker DNA sizes of different proteins of a latent variable may be predicted from comparing size differences between peaks in multimodal latent variables. Moreover, cell-free DNA degradation kinetics can be predicted from measuring relative proportions of short and long (e.g., mono-nucleosome and di-nucleosome associated, respectively) size densities of multimodal latent variables, and, the rates of change of long to short sequence-sizes along curves in latent variable space.

At step 208, one or more fragmentomic signature amplitudes of the biological sample can be determined based on projecting the set of sequence-size values (e.g., the size-distribution data) to the fragmentomic signature. The fragmentomic signature amplitudes can be determined by projecting the size-distribution data (e.g., PMFs) to the subset of latent variables of the fragmentomic signature.

At step 210, the fragmentomic signature amplitudes can be used as input to a machine-learning algorithm (e.g., a logistic-regression classifier model) to generate a result. The result can be predictive of a classification predictive of whether the subject has a particular disease. The particular disease can include cancer. In some instances, if the reference samples were obtained from the same subject but at different time points, the result is predictive of a progression or relapse of the particular disease. The result can be used to identify a treatment for the subject and/or determine how often the treatment should be administered to the subject. In another example, the result can be predictive of a presence of a chromatosome binding at, and silencing of, an allele of interest based on an algorithm determining significant enrichment of a mono- and di-chromatosome sequences associated with the allele. Accordingly, by leveraging cell-free sequence data generated from whole exome sequencing or certain genomic regions of the subject (for example), each genomic region of the set of genomic regions can be modeled as a linear mixture of allele epigenetic states (e.g., transcribed or suppressed loci) each with a different DNA sequence-size signature and amplitude in the mixture.

At step 214, the result can be outputted. For example, the result can be locally presented or transmitted to another device. The result can be outputted along with an identifier of the subject. Process 200 terminates thereafter.

### II. Sequence Data

### A. Subjects and Samples

To determine the fragmentomic signatures based on sizes of nucleic acid molecules of a biological sample of a subject, nucleic acid sequence data that represent a plurality of nucleic acid molecules can be obtained from a biological sample of a subject. The subject can be human. The subject may be a male or a female. The subject may be a fetus, infant, child, adolescent, teenager or adult. The subject may be patients of any age. For example, the subject may be a patient of less than about 10 years old. For example, the subject may be a patient of at least about 0, 5, 10, 20, 30, 40, 50, 60, 70, 80, 90, or 100 years old. The subject can be a patient or other individual undergoing a treatment regimen, or being evaluated for a treatment regimen (e.g., cancer therapy). However, in some instances, the subject is not undergoing a treatment regimen.

In some instances, the subjects may be mammals or non-mammals. In some instances, the subjects are a mammal, such as, a human, non-human primate (e.g., apes, monkeys, chimpanzees), cat, dog, rabbit, goat, horse, cow, pig, rodent, mouse, SCID mouse, rat, guinea pig, or sheep. In some embodiments, species variants or homologs of these genes are used in a non-human animal model. Species variants may be genes of different species having the greatest sequence identity and similarity in functional properties to one another. Many of such species variants human genes may be listed in a Swiss-Prot database.

Certain embodiments may include obtaining a sample from a subject, such as a human subject. In some instances, a clinical specimen from a patient is obtained. For example, blood may be drawn from a patient. Certain embodiments may include specifically detecting, profiling, or quantitating molecules (e.g., nucleic acids, DNA, RNA, etc.) that are within the biological samples.

The sample may be a tissue sample or a bodily fluid. In some instances, the sample is a tissue sample or an organ sample, such as a biopsy. In some instances, the sample includes cancerous cells. In some instances, the sample includes cancerous and normal cells. In some instances, the sample is a tumor biopsy. The bodily fluid may be sweat, saliva, tears, urine, blood, menses, semen, and/or spinal fluid. In some instances, the sample is a blood sample. The sample may include one or more peripheral blood lymphocytes. The sample may be a whole blood sample. The blood sample may be a peripheral blood sample. In some instances, the sample includes peripheral blood mononuclear cells (PBMCs); in some cases, the sample includes peripheral blood lymphocytes (PBLs). The sample may be a serum sample. The sample may be a plasma sample.

The sample may be obtained using any method that can provide a sample suitable for the analytical methods described herein. The sample may be obtained by a non-invasive method such as a throat swab, buccal swab, bronchial lavage, urine collection, scraping of the skin or cervix, swabbing of the cheek, saliva collection, feces collection, menses collection, or semen collection. The sample may be obtained by a minimally-invasive method such as a blood draw. The sample may be obtained by venipuncture. In other instances, the sample is obtained by an invasive procedure including but not limited to: biopsy, alveolar or pulmonary lavage, or needle aspiration. The method of biopsy may include surgical biopsy, incisional biopsy, excisional biopsy, punch biopsy, shave biopsy, or skin biopsy. The sample may be formalin fixed sections. The method of needle aspiration may further include fine needle aspiration, core needle biopsy, vacuum assisted biopsy, or large core biopsy. In some instances, multiple samples may be obtained by the methods herein to ensure a sufficient amount of biological material. In some instances, the sample is not obtained by biopsy.

### B. Generating the Sequence Data

In some embodiments, the sample is processed to obtain nucleic acid sequence data. “Nucleic acid” or “nucleic acid molecules” can correspond to a polymeric form of nucleotides of any length, either ribonucleotides, deoxyribonucleotides or peptide nucleic acids (PNAs), that include purine and pyrimidine bases, or other natural, chemically or biochemically modified, non-natural, or derivatized nucleotide bases. The backbone of the polynucleotide can include sugars and phosphate groups, as may typically be found in RNA or DNA, or modified or substituted sugar or phosphate groups. A polynucleotide may include modified nucleotides, such as methylated nucleotides and nucleotide analogs. The sequence of nucleotides may be interrupted by non-nucleotide components. Thus, the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. The nucleic acid molecule may be a DNA molecule. The nucleic acid molecule may be an RNA molecule.

The sample processing includes nucleic acid sample processing and subsequent nucleic acid sample sequencing. Some or all of the biological sample may be sequenced to provide the nucleic acid sequence data, which may be stored or otherwise maintained in an electronic, magnetic or optical storage location. The sequence information may be analyzed with the aid of a computer processor, and the analyzed sequence information may be stored in an electronic storage location. The electronic storage location may include a pool or collection of sequence information and analyzed sequence information generated from the nucleic acid sample. In some embodiments, the biological sample is retrieved from a subject that has or is suspected of having cancer.

In some embodiments, nucleic acid sequence data are generated from pure tumor and pure normal samples. Matched pair cell lines can be obtained from another source (e.g., American Type Culture Collection). Each matched pair may include a tumor cell line and a normal cell line from the same subject. The cell lines can be cultured and expanded in vitro to obtain a suitable number of cells for DNA extraction. DNA is extracted, processed, and subjected to whole exome or whole genome sequencing. Sequence reads can be subjected to quality control processing (e.g., via FastQC) to provide FASTQ files.

In some instances, the nucleic acid sequence data is generated using whole genome sequencing. In some instances, the whole genome sequencing is used to identify variants in a person. The whole genome sequencing can include a shallow sequencing (1-5× coverage) or a deep sequencing (30-100× coverage) over an entirety or nearly the entirety of the genome. In some instances, sequencing includes sequencing over a fraction of the genome. For example, the fraction of the genome may be at least about 50; 75; 100; 125; 150; 175; 200; 225; 250; 275; 300; 350; 400; 450; 500; 550; 600; 650; 700; 750; 800; 850; 900; 950; 1,000; 1100; 1200; 1300; 1400; 1500; 1600; 1700; 1800; 1900; 2,000; 3,000; 4,000; 5,000; 6,000; 7,000; 8,000; 9,000; 10,000; 15,000; 20,000; 30,000; 40,000; 50,000; 60,000; 70,000; 80,000; 90,000; 100,000 or more bases or base pairs. In some instances, the genome may be sequenced over 1 million, 2 million, 3 million, 4 million, 5 million, 6 million, 7 million, 8 million, 9 million, 10 million or more than 10 million bases or base pairs. In some instances, the genome may be sequenced over an entire exome (e.g., whole exome sequencing). In some instances, the deep sequencing may include acquiring multiple reads over the fraction of the genome. For example, acquiring multiple reads may include at least 2, 3, 4, 5, 6, 7, 8, 9, 10, 20, 30, 40, 50, 60, 70, 80, 90, 100, 200, 300, 400, 500, 600, 700, 800, 900, 1000, 10,000 reads or more than 10,000 reads over the fraction of the genome.

Additionally or alternatively, the nucleic acid sequence data can be generated using panel-based sequencing. Panel-based sequencing can be used to simultaneously assess multiple potential genetic causes of a suspected disorder, disease, or a phenotype. Gene panels can be used to identify targeted genomic regions, to which sequence nucleic acid molecules align. In some instances, a number of targeted genomic regions include at least 50, 100, 200, 500, 1000, 1500, 2000 genomic regions. Instead of the number of targeted genomic regions, panel-based sequencing can define a footprint for the targeted genomic regions. For example, the footprint may range between 175 Kb and 3 Gb. In other embodiments, the panel-based sequencing can target a number of genomic regions of known biological significance, such as genomic regions associated with variants in cancer driver or tumor escape genes.

In some instances, generating the nucleic acid sequence data includes detecting low allelic fractions by deep sequencing. In some instances, the deep sequencing is done by next generation sequencing. In some instances, the deep sequencing is performed by avoiding error-prone regions. In some instances, the error-prone regions may include regions of near sequence duplication, regions of unusually high or low % GC, regions of near homopolymers, di- and tri-nucleotide, and regions of near other short repeats. In some instances, the error-prone regions may include regions that lead to DNA sequencing errors (e.g., polymerase slippage in homopolymer sequences).

In some instances, generating the nucleic acid sequence data includes conducting one or more sequencing reactions on one or more nucleic acid molecules in a sample. Certain embodiments may include conducting 1 or more, 2 or more, 3 or more, 4 or more, 5 or more, 6 or more, 7 or more, 8 or more, 9 or more, 10 or more, 15 or more, 20 or more, 30 or more, 40 or more, 50 or more, 60 or more, 70 or more, 80 or more, 90 or more, 100 or more, 200 or more, 300 or more, 400 or more, 500 or more, 600 or more, 700 or more, 800 or more, 900 or more, or 1000 or more sequencing reactions on one or more nucleic acid molecules in a sample. The sequencing reactions may be run simultaneously, sequentially, or a combination thereof. The sequencing reactions may include whole genome sequencing, exome sequencing or smaller panel targeted sequencing. The sequencing reactions may include Maxim-Gilbert, chain-termination or high-throughput systems. Alternatively, or additionally, the sequencing reactions may include Helioscope™ single molecule sequencing, Nanopore DNA sequencing, Lynx Therapeutics' Massively Parallel Signature Sequencing (MPSS), 454 pyrosequencing, Single Molecule real time (RNAP) sequencing, Illumina (Solexa) sequencing, SOLiD sequencing, Ion Torrent™, Ion semiconductor sequencing, Single Molecule SMRT™ sequencing, Polony sequencing, DNA nanoball sequencing, VisiGen Biotechnologies approach, or a combination thereof. Alternatively, or additionally, the sequencing reactions can include one or more sequencing platforms, including, but not limited to, Genome Analyzer Ix, HiSeq, MiSeq and NovaSeq offered by Illumina, Single Molecule Real Time (SMRT™) technology, such as the PacBio RS system offered by Pacific Biosciences (California) and the Solexa Sequencer, True Single Molecule Sequencing (tSMS™) technology such as the HeliScope™ Sequencer offered by Helicos Inc. (Cambridge, MA). Sequencing reactions may also include electron microscopy or a chemical-sensitive field effect transistor (chemFET) array. In some aspects of the disclosure, sequencing reactions include capillary sequencing, next generation sequencing, Sanger sequencing, sequencing by synthesis, sequencing by ligation, sequencing by hybridization, single molecule sequencing, or a combination thereof. Sequencing by synthesis may include reversible terminator sequencing, processive single molecule sequencing, sequential flow sequencing, or a combination thereof. Sequential flow sequencing may include pyrosequencing, pH-mediated sequencing, semiconductor sequencing, or a combination thereof.

In some instances, generating the nucleic acid sequence data includes conducting at least one long read sequencing reaction and at least one short read sequencing reaction. The long read sequencing reaction and/or short read sequencing reaction may be conducted on at least a portion of a subset of nucleic acid molecules. The long read sequencing reaction and/or short read sequencing reaction may be conducted on at least a portion of two or more subsets of nucleic acid molecules. Both a long read sequencing reaction and a short read sequencing reaction may be conducted on at least a portion of one or more subsets of nucleic acid molecules.

Sequencing of the one or more nucleic acid molecules or subsets thereof may include at least about 5; 10; 15; 20; 25; 30; 35; 40; 45; 50; 60; 70; 80; 90; 100; 200; 300; 400; 500; 600; 700; 800; 900; 1,000; 1,500; 2,000; 2,500; 3,000; 3,500; 4,000; 4,500; 5,000; 5,500; 6,000; 6,500; 7,000; 7,500; 8,000; 8,500; 9,000; 9,500; 10,000; 25,000; 50,000; 75,000; 100,000; 250,000; 500,000; 750,000; 10,000,000; 25,000,000; 50,000,000; 100,000,000; 250,000,000; 500,000,000; 750,000,000; 1,000,000,000 or more sequencing reads.

Sequencing reactions may include sequencing at least about 50; 60; 70; 80; 90; 100; 110; 120; 130; 140; 150; 160; 170; 180; 190; 200; 210; 220; 230; 240; 250; 260; 270; 280; 290; 300; 325; 350; 375; 400; 425; 450; 475; 500; 600; 700; 800; 900; 1,000; 1,500; 2,000; 2,500; 3,000; 3,500; 4,000; 4,500; 5,000; 5,500; 6,000; 6,500; 7,000; 7,500; 8,000; 8,500; 9,000; 10,000; 20,000; 30,000; 40,000; 50,000; 60,000; 70,000; 80,000; 90,000; 100,000 or more bases or base pairs of one or more nucleic acid molecules. Sequencing reactions may include sequencing at least about 50; 60; 70; 80; 90; 100; 110; 120; 130; 140; 150; 160; 170; 180; 190; 200; 210; 220; 230; 240; 250; 260; 270; 280; 290; 300; 325; 350; 375; 400; 425; 450; 475; 500; 600; 700; 800; 900; 1,000; 1,500; 2,000; 2,500; 3,000; 3,500; 4,000; 4,500; 5,000; 5,500; 6,000; 6500; 7,000; 7,500; 8,000; 8,500; 9,000; 10,000; 20,000; 30,000; 40,000; 50,000; 60,000; 70,000; 80,000; 90,000; 100,000 or more consecutive bases or base pairs of one or more nucleic acid molecules.

In some instances, the sequencing technique generates at least 100 reads per run, at least 200 reads per run, at least 300 reads per run, at least 400 reads per run, at least 500 reads per run, at least 600 reads per run, at least 700 reads per run, at least 800 reads per run, at least 900 reads per run, at least 1,000 reads per run, at least 5,000 reads per run, at least 10,000 reads per run, at least 50,000 reads per run, at least 100,000 reads per run, at least 500,000 reads per run, or at least 1,000,000 reads per run. Alternatively, the sequencing technique generates at least 1,500,000 reads per run, at least 2,000,000 reads per run, at least 2,500,000 reads per run, at least 3,000,000 reads per run, at least 3,500,000 reads per run, at least 4,000,000 reads per run, at least 4,500,000 reads per run, or at least 5,000,000 reads per run.

In some instances, the sequencing technique generates at least about 30 base pairs, at least about 40 base pairs, at least about 50 base pairs, at least about 60 base pairs, at least about 70 base pairs, at least about 80 base pairs, at least about 90 base pairs, at least about 100 base pairs, at least about 110, at least about 120 base pairs per read, at least about 150 base pairs, at least about 200 base pairs, at least about 250 base pairs, at least about 300 base pairs, at least about 350 base pairs, at least about 400 base pairs, at least about 450 base pairs, at least about 500 base pairs, at least about 550 base pairs, at least about 600 base pairs, at least about 700 base pairs, at least about 800 base pairs, at least about 900 base pairs, or at least about 1,000 base pairs per read. Additionally, or alternatively, the sequencing technique can generate long sequencing reads. In some instances, the sequencing technique can generate at least about 1,200 base pairs per read, at least about 1,500 base pairs per read, at least about 1,800 base pairs per read, at least about 2,000 base pairs per read, at least about 2,500 base pairs per read, at least about 3,000 base pairs per read, at least about 3,500 base pairs per read, at least about 4,000 base pairs per read, at least about 4,500 base pairs per read, at least about 5,000 base pairs per read, at least about 6,000 base pairs per read, at least about 7,000 base pairs per read, at least about 8,000 base pairs per read, at least about 9,000 base pairs per read, at least about 10,000 base pairs per read, at least about 20,000 base pairs per read, at least about 30,000 base pairs per read, at least about 40,000 base pairs per read, at least about 50,000 base pairs per read, at least about 60,000 base pairs per read, at least about 70,000 base pairs per read, at least about 80,000 base pairs per read, at least about 90,000 base pairs per read, or at least about 100,000 base pairs per read.

High-throughput sequencing systems may allow detection of a sequenced nucleotide immediately after or upon its incorporation into a growing strand, i.e., detection of sequence in real time or substantially real time. In some instances, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120, at least 150, at least 200, at least 250, at least 300, at least 350, at least 400, at least 450, or at least 500 bases per read. Sequencing can be performed using nucleic acids described herein such as genomic DNA, mtDNA, cDNA derived from RNA transcripts or RNA as a template.

### III. Input Data for Determining Latent Variables

The fragmentomic signature can be defined to include one or more latent variables. Each latent variable can identify an estimated size and/or end-motif distribution of sequences that is variably enriched across the set of genomic regions. The latent variables can represent a new independent basis vector for sequence-size and/or end-motif data, which are then used to determine the enrichment of fragmentomic signatures of the subject.

### A. Sequence-Size Values

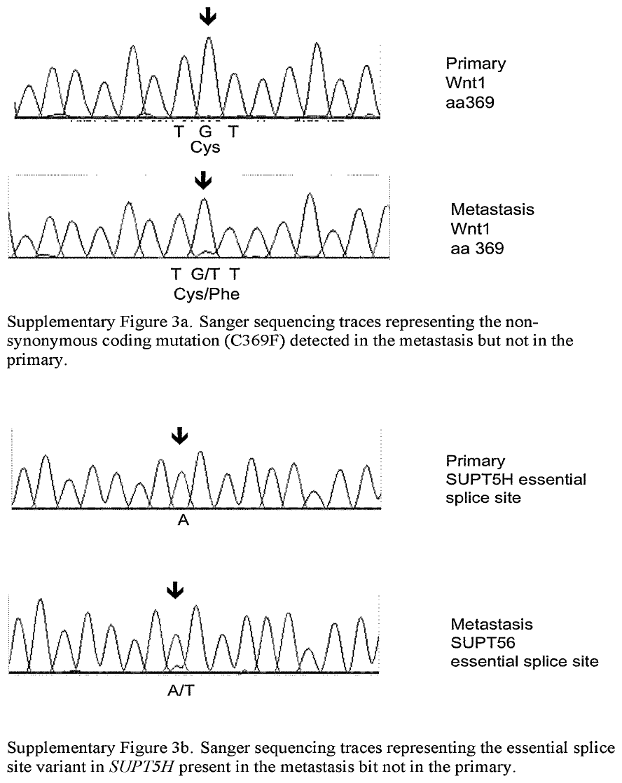

FIG. 3 shows a schematic diagram 300 that illustrates an example technique for generating sequence-size values, according to some embodiments. The schematic diagram 300 shows sequence data 305 that are aligned to a reference sequence 310. In some instances, reference sequence 310 is a human reference genome (e.g., a hg19 genome). The reference sequence 310 can be divided into a set of genomic regions, including a genomic region 315. In the example presented in FIG. 3, a set of sequences of the sequence data 305 can align to the genomic region 315. For the genomic region 315, a size distribution of the set of sequences can be identified.

Based on the sequence data 305, a plurality of sets of sequence-size values 320 can be generated. Each set of sequence-size values can include, for each genomic region, sequence-size values corresponding to sizes of a corresponding set of sequences. A set of sequence-size values can correspond to a set of integers within a rectangle. In some instances, a set of sequence-size values across the whole genome is used. The set of sequence-size values can be determined from sequences aligning to a set of genomic regions (e.g., genomic regions at which somatic variants are known to be associated with cancer).

In some instances, each sequence represented by a corresponding sequence-size value includes a DNA fragment having a size ranging between 50 bp and 550 bp. Sequence-size values (e.g., template fragment-length values in SAM/BAM files) outside the range between 50 and 550 bp were not used in the analysis.

In addition to or as an alternative of the sets of sequence-size values, each set of sequence-size values can be used to generate a PMF 325. The PMF can refer to as a function that gives the probability that a discrete random variable (e.g., a sequence-size value) is equal to some value (e.g., 167 bp) within a particular range.

In some embodiments, the PMF 325 is generated for each set of sequence-size values, in which each sequence-size value of the set was binned at 1 bp resolution then normalized by total read pair count. The set of PMFs were used as input data (e.g., a matrix that includes N×501 dimensions) for a BSS algorithm to identify one or more components that are predictive of a particular disease.

### B. End Motifs

An end motif can be identified as an ending sequence of nucleotides of a DNA fragment, e.g., sequence for the K bases at either end of the fragment. The ending sequence can be a k-mer having various numbers of bases, e.g., 1, 2, 3, 4, 5, 6, 7. In some instances, the end motif is determined by aligning the sequence reads to a reference genome and identify nucleotide bases just before a start position or just after an end position. Such bases will still correspond to ends of the DNA fragments, e.g., as they are identified based on the ending sequences of the fragments.

The end motifs can be identified from the aligned sequence reads using various techniques. In some instances, the k-mer end motifs are directly constructed from the first k-bp sequence on each end of a plasma DNA molecule. For example, the first 4 nucleotides or the last 4 nucleotides of a sequenced fragment could be used. In another example, the k-mer end motifs are jointly constructed by making use of the (k−2)-mer sequence from the sequenced ends of fragments and the other (k−2)-mer sequence from the genomic regions adjacent to the ends of that fragment. Various lengths of end motifs can be used, e.g., 1-mer, 2-mer, 3-mer, 5-mer, 6-mer, 7-mer end motifs. The sequence reads can be aligned to the end-motifs, thereby generating a set of end-motif sequence data. The set of end-motif sequence data can be applied to one or more signal-separation algorithms to generate a corresponding set of latent variables.

The higher the number of nucleotides included in the cell-free DNA end signature, the higher the specificity of the motif. For example, a probability of having 6 bases ordered in an exact configuration in the genome is lower than the probability of having 2 bases ordered in an exact configuration in the genome. Thus, the choice of the length of the end motif can be governed by the needed sensitivity and/or specificity of the intended use application.

### IV. Determining Latent Variables of a Fragmentomic Signature Using Signal-Separation Algorithms

To determine latent variables of fragmentomic signatures, one or more signal-separation algorithms can be applied to the plurality of sets of sequence-size values and/or end-motif sequence data. In some instances, each latent variable of the set of latent variables includes a histogram or a weight vector that represents a size distribution underlying a proportion of the sequence data for the biological sample. The one or more signal-separation algorithms can be a blind-source separation algorithm that can use an independent component analysis algorithm and/or a non-negative matrix factorization algorithm.

### A. Signal-Separation Algorithms

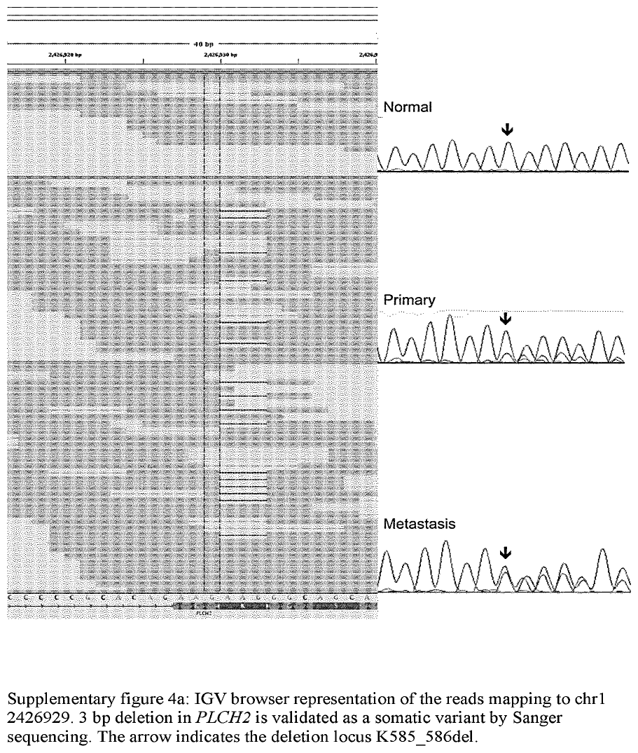

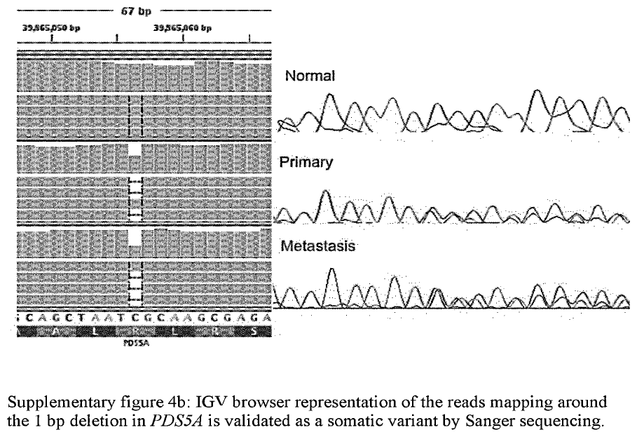

Signal-separation algorithms can be configured and employed to estimate a set of source signals from a set of observed signals, in which each observed signal is a mixture of source signals. FIG. 4 shows an example diagram 400 illustrating signal-separation algorithms being applied to sets of image mixtures made from linearly summing image source signals. In FIG. 4, input images 405 were generated based on a mixture of a set of image signals 410. Each image signal S, can be scaled by a random coefficient k, to generate source image amplitude diversity in the image mixture compendium. At least one image signal can include a unique random noise signal per image mixture. In FIG. 4, the two thousand input images 405 constructed based on a mixture of a set of four image signals 410 can be represented by the following expression:

$${\sum }_{1}^{4} {k}_{i} {s}_{i} + 10 {s}_{n o i s e}$$

The input images 405 can be processed using one or more signal-separation algorithms to output a set of images 415 that visually approximate the images represented by the set of image signals 410. The signal-separation algorithm can be a blind-source separation algorithm. The blind-source separation algorithm is referred to as “blind” since the algorithm can estimate the source signals without directly observing them but instead by observing their influence on measurable elementary quantities.

The blind-source separation algorithm can include a principal component analysis (PCA) achieved by a Singular Value Decomposition (SVD) algorithm 420 or an ICA algorithm 430 (e.g., fastICA, InfoMax). Both of the SVD algorithm 420 and the ICA algorithm 430 can be trained on a set of data, and their outputs can be applied to new sets of data. As used herein, the SVD algorithm 420 can refer to a technique for identifying a smaller number of uncorrelated variables known as principal components from a larger set of data. The technique is widely used to emphasize variation and capture strong patterns in a data set (e.g., a set of pixels corresponding to the input image 405). In FIG. 4, an example set of unmixed images 425 can be shown as outputs generated by applying the SVD algorithm 420 to the entire set of two thousand image mixtures 405. In some instances, the input images 405 are pre-processed before applying the SVD algorithm 420.

The ICA algorithm 430 can include a fixed-point iteration scheme for finding a maximum of non-Gaussianity, at which the ICA algorithm can be iteratively executed until the maximum non-Gaussian value is reached. In some instances, the input images 405 are pre-processed before applying the ICA algorithm 430 including zero centering and scaling to unit variance. As shown in FIG. 4, example sets of unmixed images 435 and 440 can be shown as outputs generated by applying the ICA algorithm 430 to different amounts of the input images 405. Visual characteristics of a first set of unmixed images 435 do not appear to match those of the set of image signals 410, likely due to use of a low number (10) of image mixtures (I₁-I₁₀) with relatively low source image amplitude diversity. By contrast, visual characteristics of a second set of images 440 appear to match those of the respective set of image signals, likely due to use of a high number (2000) of image mixtures (I₁-I₂₀₀₀) with an abundance of source image amplitude diversity. In addition, although not shown in FIG. 4, the blind-source separation algorithm can include an NMF algorithm.

### B. Example Scheme for Determining the Latent Variables of a Fragmentomic Signature

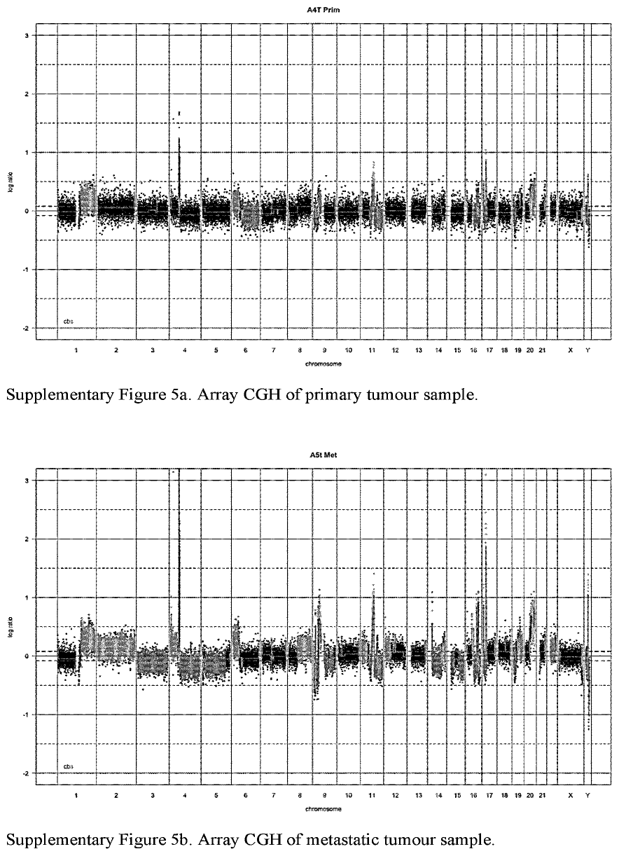

FIG. 5 shows a schematic diagram 500 that illustrates an example technique for generating a set of latent variables, according to some embodiments. As shown in FIG. 5, each set the plurality of sets of sequence-size values 505 can be used as the input for a BSS algorithm 510. The BSS algorithm 510 can include an ICA algorithm 515 or an NMF algorithm 520. Depending on a type of the BSS algorithm, additional formatting of the plurality of sets of sequence-size values 505 can be performed. For example, the NMF algorithm 520 can use raw PMFs that correspond to the sets of sequence-size values. In another example, the ICA algorithm 515 can use PMFs that after they are first mean centered then scaled to unit variance. In some instances, the BSS algorithm 510 is executed multiple times until stochastic initial states achieved convergence (e.g., solution reached, minimization or maximization criteria met, maximized non-Gaussianity for ICA). The repeated executions of BSS algorithms were performed to insure capture of well-estimated latent variables from repeatability across multiple runs.

Although the example technique of FIG. 5 discusses generating a set of latent variables from the sets of sequence-size values, the BSS algorithm 510 can also be applied to a set of end-motif sequence data to generate another set of latent variables. In this case, the other set of latent variables identify end-motif distributions of the sequence data. For example, the end-motif distributions can include, for each k-mer end motif of nucleotides (e.g., CCCA, TAAA), a number or relative frequency of DNA fragments having an ending sequence that correspond to the k-mer end motif. By separating the end-motif distributions of sequence data into a set of independent weight vectors, the fragmentomic signature can be determined.

Additionally or alternatively, the BSS algorithm 510 can also be applied to generate the set of latent variables, such that each latent variable can identify an estimated size distribution of sequence reads that have ending sequences corresponding to a respective end motif.

The input to the ICA algorithm 515 is a data matrix X and the output includes: (i) an S matrix that includes histogram or signed weight vector data corresponding to the set of latent variables; and (ii) an A linear mixing matrix that includes estimated amplitudes of latent variables across the set of genomic regions. Similarly, the input to the NMF algorithm 520 is a data matrix Y and the output includes: (i) a W matrix that includes the non-negative histogram data corresponding to the set of latent variables; and (ii) an H matrix that includes estimated amplitudes of latent variables across the set of genomic regions. These data matrices can be used to generate a set of histograms or weight vectors, which can be represented as latent variables 525 of a corresponding fragmentomic signature.

Continuing with the examples shown in FIG. 5, the latent variables 525 correspond to weight vectors or histograms generated by applying the BSS algorithm 510 to the plurality of sets of sequence-size values 505. The peaks and amplitudes identified in each of the latent variables 525 can reveal size of nucleic acid molecules of a biological sample and/or sequence-fragmentation pattern of sequences in the sequence data. The latent variables 525 can represent a fragmentomic signature of the subject.

### V. Using Latent Variables of Fragmentomic Signatures to Detect Cancer-Related Gene Mutations

Many cancers are caused by recurrent gene mutations, such as BRCA and HER2 mutations for breast cancers. Therefore, accurate detection of these cancer-related gene mutations is the key for target therapy for cancer patients. However, noises can be produced in various steps of NGS experiments, such as PCR errors or sequencing errors. These noises are especially problematic for cfDNA samples, where the allele frequency for real mutation is low. Previous studies show that cfDNA molecules from cancer tissue have different fragment size distribution compared with cfDNA molecules from healthy tissue. By investigating fragmentomic signatures of mutation-supporting reads, we can confirm whether the observed mutations are cancer-related or noise-related.

A. Generate Fragmentomic Signatures from a Set of cfDNA Whole Exome Sequencing (WES) Samples

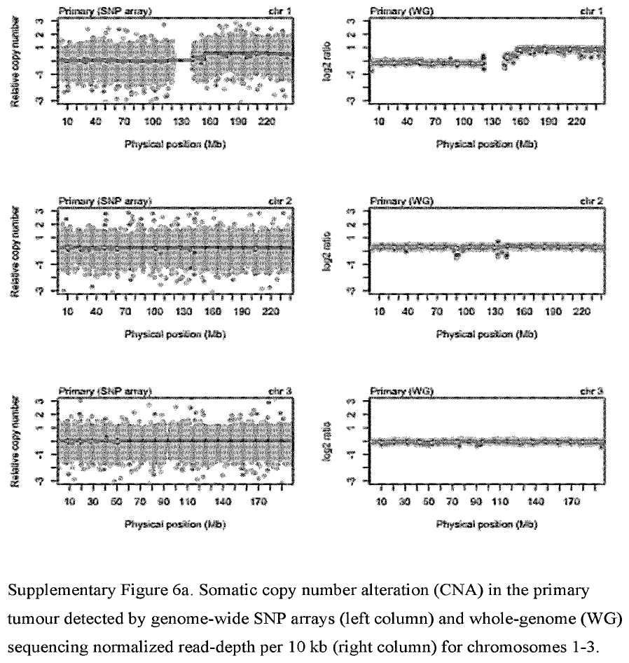

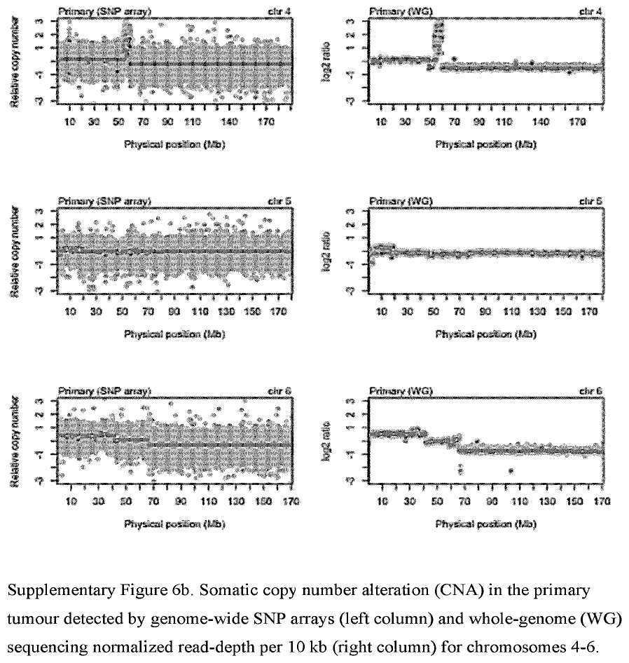

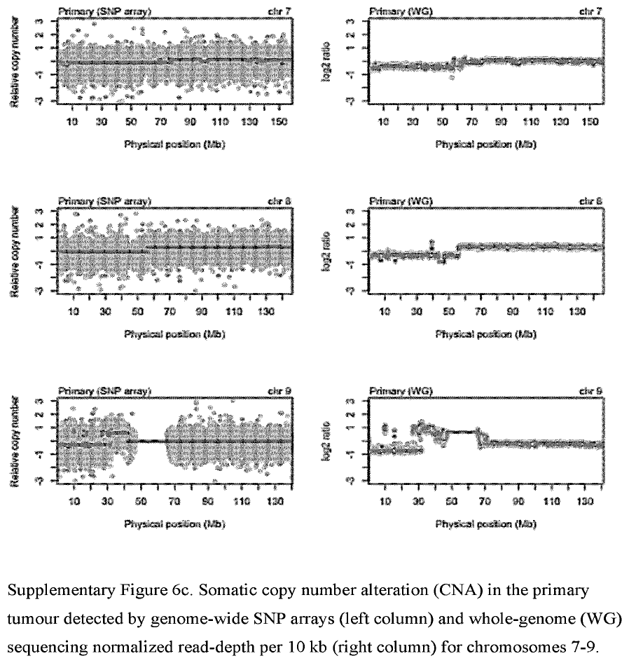

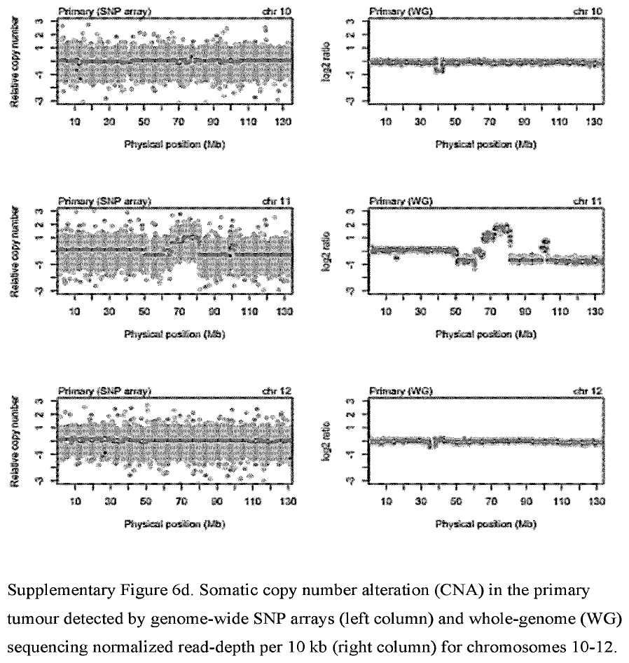

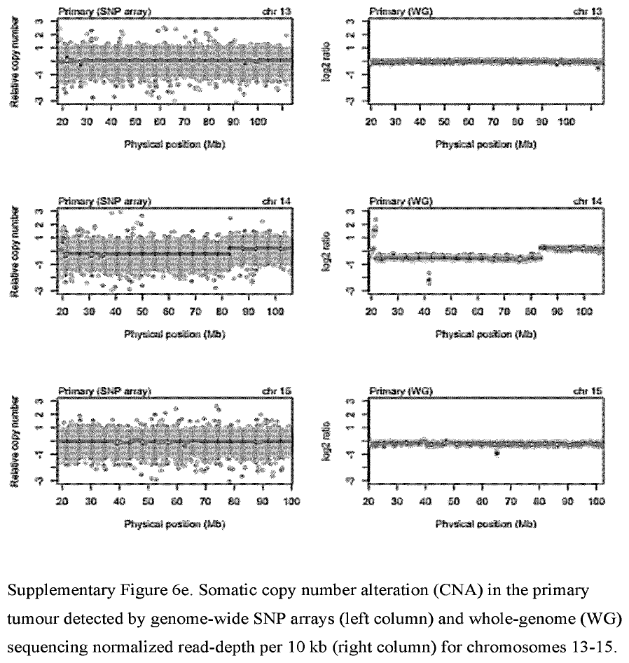

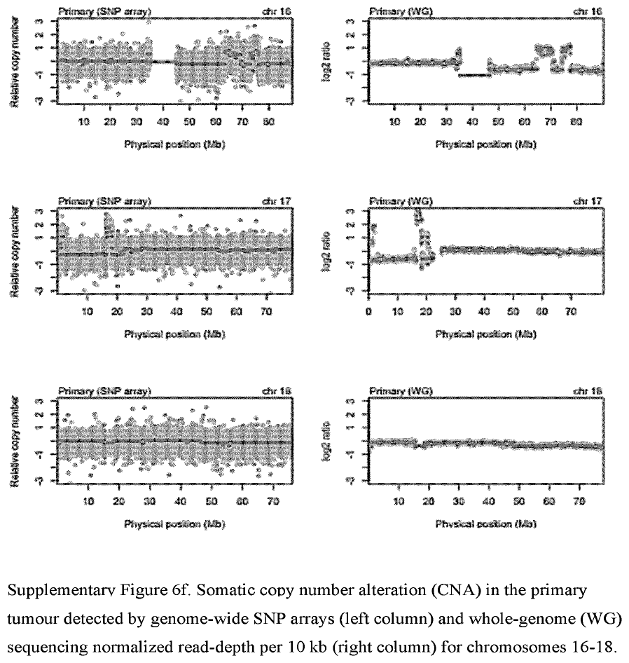

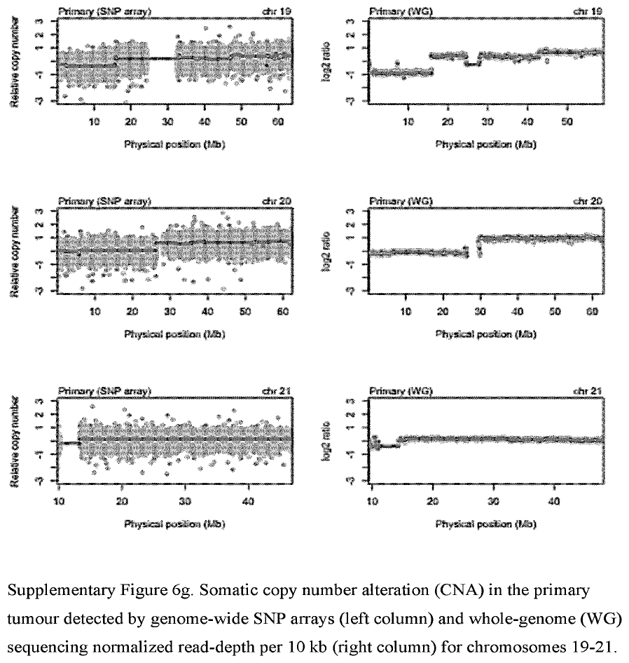

FIG. 6 shows a schematic diagram 600 that illustrates an example technique of using independent component analysis algorithm for generating a set of latent variables for a reference signature, according to some embodiments. As shown in FIG. 6, a set of cfDNA samples 605 (n=10) can be collected. The set of cfDNA samples 605 can include 4 colorectal cancer samples cfDNA identified by a prefix “CRC” and 6 healthy donor samples identified by a prefix “PON”. The set of biological samples 605 can be sequenced using Whole Exome Sequencing (WES) to generate the sequence data, from which sets of sequence-size values can be generated.

The sequence data is divided into 309 uniform intervals, and the fragment size distribution of each region can be generated. The ICA algorithm is then applied to the 309×541 (60-600 bp) matrix of each sample 610 to generate S matrix corresponding to a set of latent variables. To select the most relevant latent variables from such set, a clustering algorithm 615 (e.g., a k-means clustering algorithm) can be applied to the set of latent variables from all samples, in which the latent variables of the set can be clustered together based on their similarities. For each cluster, a latent variable centroid can be generated. For each subject, the latent variables that are most similar to the cluster centroids can be used for detecting cancer mutations. For example, latent variables 620 similar to cluster centroids can be selected from a healthy cfDNA sample. In another example, latent variables 625 similar to cluster centroids can be selected from a cfDNA sample that is associated with colorectal cancer.

### B. Comparing the Latent Variables Between Healthy Donors and Cancer Patients

Referring back to FIG. 6, latent variables 620 of nucleic acid molecules of a normal biological sample is compared with latent variables 625 of nucleic acid molecules of a biological sample with tumor DNA (e.g., TP53 tumor variant). The differences between the latent variables 620 and the latent variables 625 can be used to predict cancer-related gene mutations.

Continuing with the examples in FIG. 6, the comparison can include comparing a sequence-size value of a peak shown in each of the set of latent variables 620 with a sequence-size value of a peak shown in a corresponding latent variable of the set of latent variables 625. In one example, the sequence-size values of peaks shown in the set of latent variables 625 (colorectal cancer samples) are approximately 10-30 base pairs less than those shown in the set of latent variables 620 (healthy samples). Thus, the abundance of relatively shorter DNA molecules can be predictive of whether the subject has cancer. In some instances, a difference between a sequence-size value of a peak of a latent variable from a cancer sample and a sequence-size value of a peak of a corresponding latent variable from a healthy sample is calculated to predict a particular stage of disease. For example, if the difference indicates a relatively small value (e.g., less than 10 bp), then it can be predicted that the cancer is at an early stage. In contrast, if the difference indicates a relatively large value (e.g., greater than 30 bp), then it can be predicted that the cancer is at a late stage.

Additionally or alternatively, the comparison can include determining, for each latent variable, a density of sequence-size values within a predetermined size range. The density of sequence-size values can be compared against a predetermined threshold, in order to predict whether the subject has a particular disease. For example, a density of sequence-size values of a latent variable within size range of 200-300 bp can be calculated, and the density can be compared against the threshold (e.g., 2) to predict whether the subject has cancer.

Additionally or alternatively, the comparison of the latent variables can include comparing an amplitude value of each latent variable of the set of latent variables 620 and an amplitude value of a corresponding latent variable of the set of latent variables 625. In some instances, a difference between each pair of amplitude values is compared to a threshold.

### C. Examples of Fragmentomic Signatures for Individual Sample

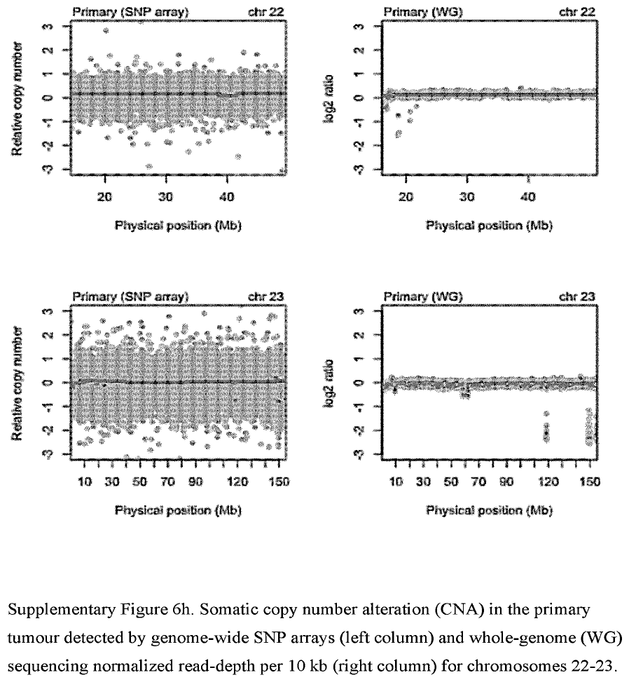

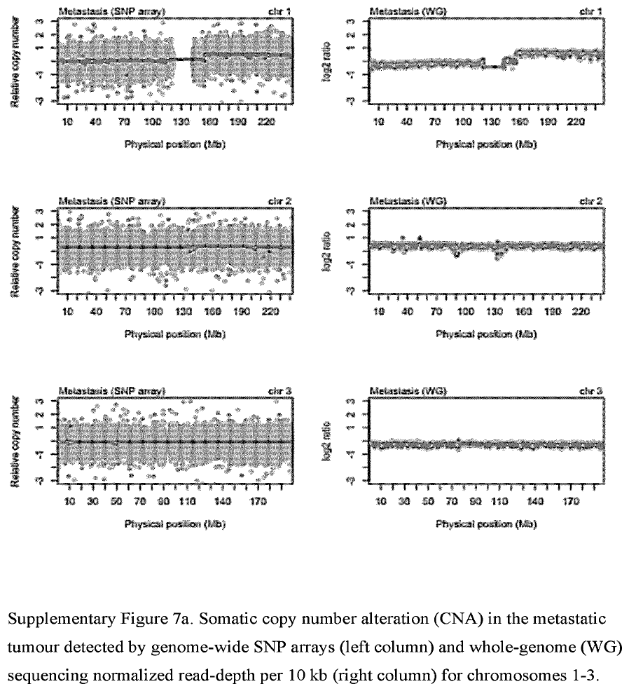

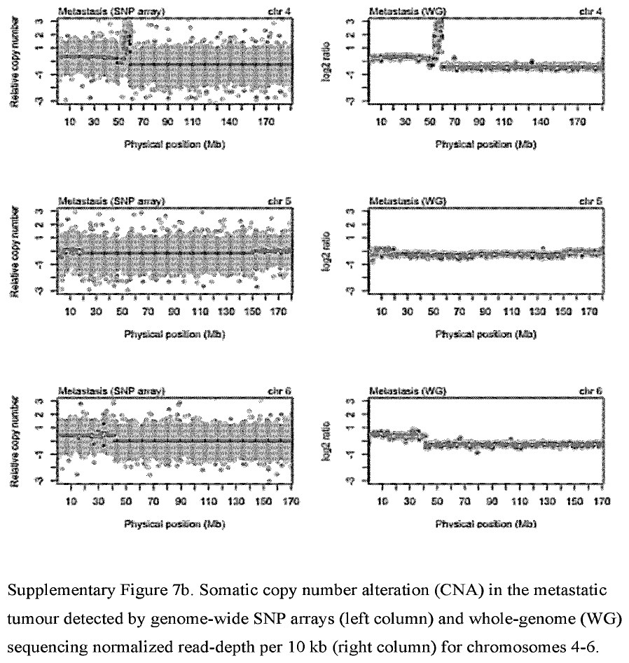

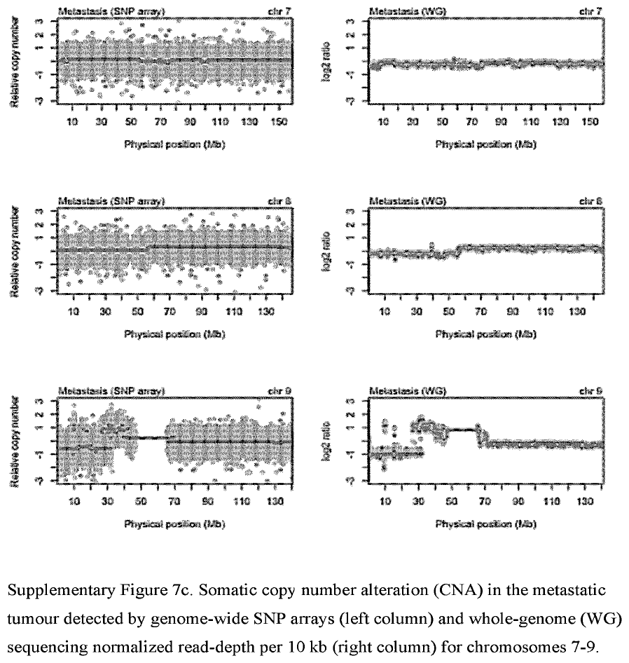

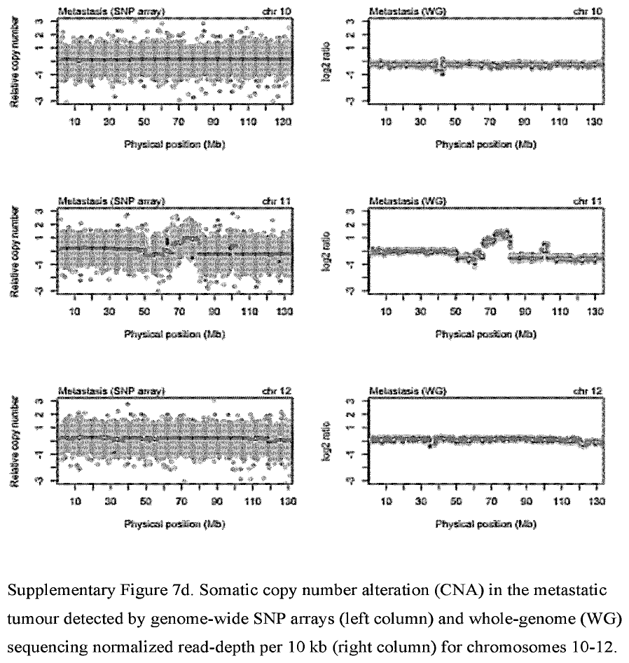

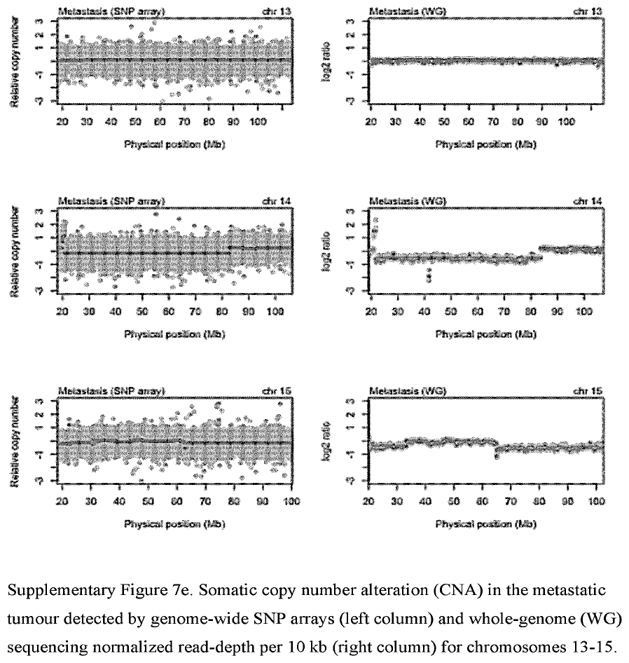

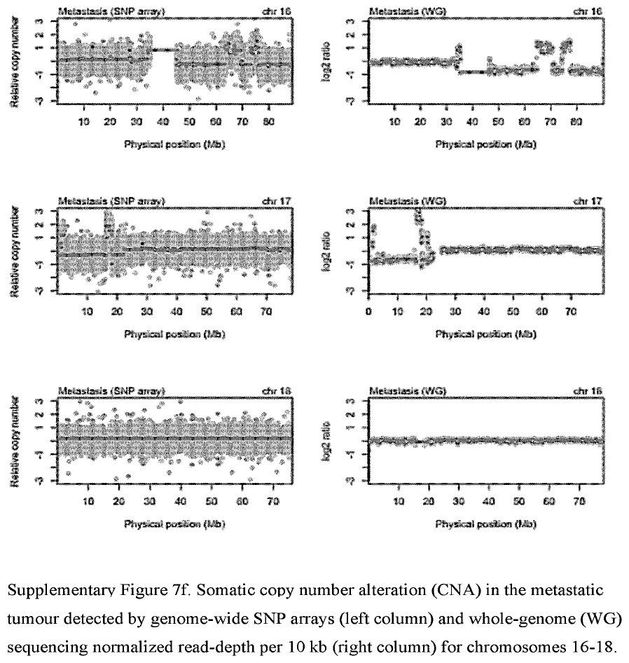

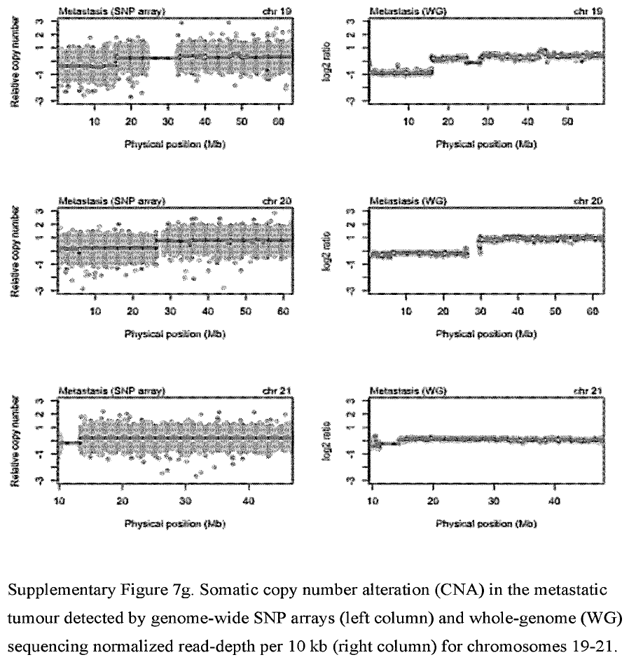

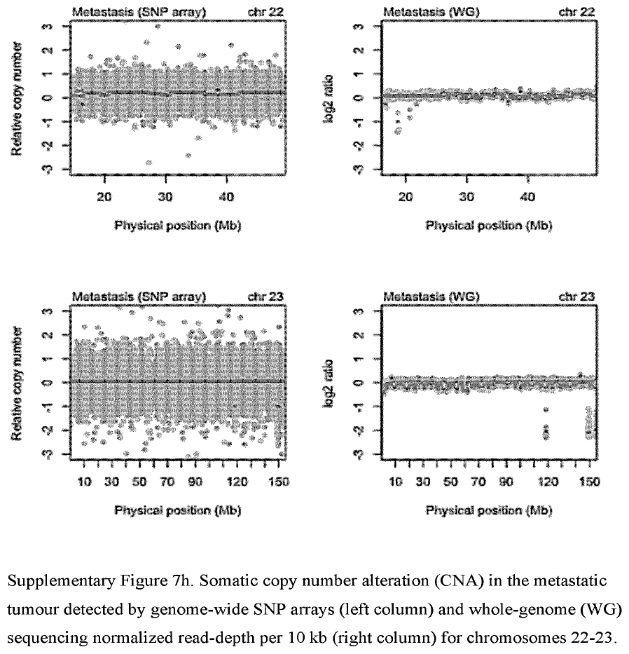

FIGS. 7-10 illustrate an example set of fragmentomic signatures for individual samples, according to some embodiments. In each figure, we highlight the latent variables that are repeatedly-identified across samples and across multiple runs of the BSS algorithm. FIG. 7 shows a set of latent variables 700 generated by applying a signal-separation algorithm on sequence-size values corresponding to a biological sample of a normal subject. A subset of latent variables 705 can be selected from the set of latent variables 700, in which the subset of latent variables 705 identify size distributions of sequences that are representative of the corresponding biological sample fragmentomic signatures. In some instances, a latent variable of the subset 705 is selected based on whether the latent variable includes a peak with peak-width value greater than a particular threshold. Additionally, or alternatively, the latent variables of the subset 705 can be selected by applying a clustering algorithm to the latent variables to generate a set of clusters and selecting a latent variable that corresponds to a centroid of a particular cluster of the set.

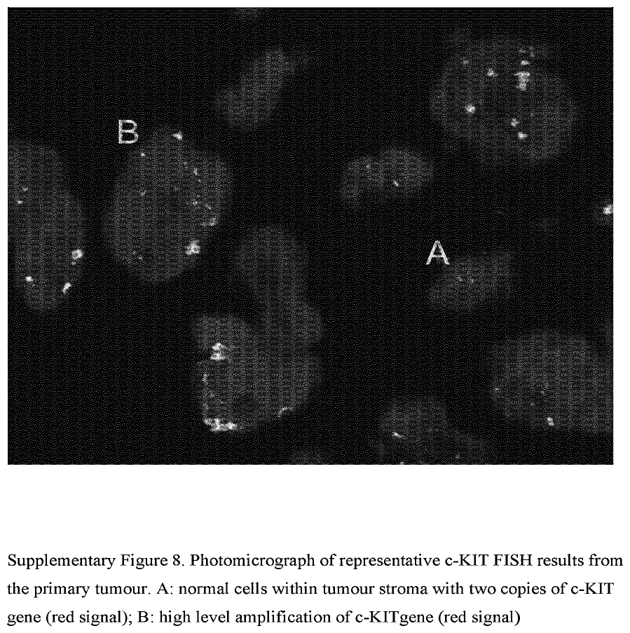

FIG. 8 shows a set of latent variables 800 generated by applying a signal-separation algorithm on sequence-size values corresponding to a biological sample of another normal subject. A subset of latent variables 805 can be selected from the set of latent variables 800, in which the subset of latent variables 805 identify size distributions of sequences that are representative of the corresponding biological sample fragmentomic signatures. As shown in FIG. 8, the subset of latent variables 805 include a subset of latent variables that share similarities with the latent variables of the fragmentomic signature 705 of FIG. 7.

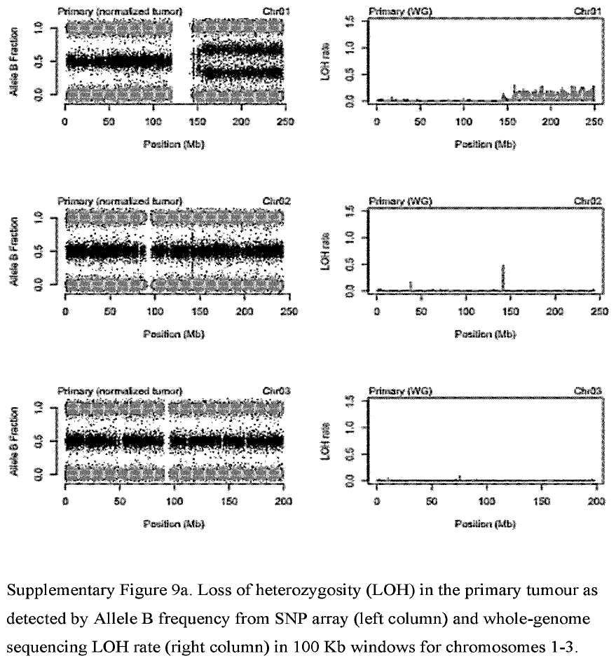

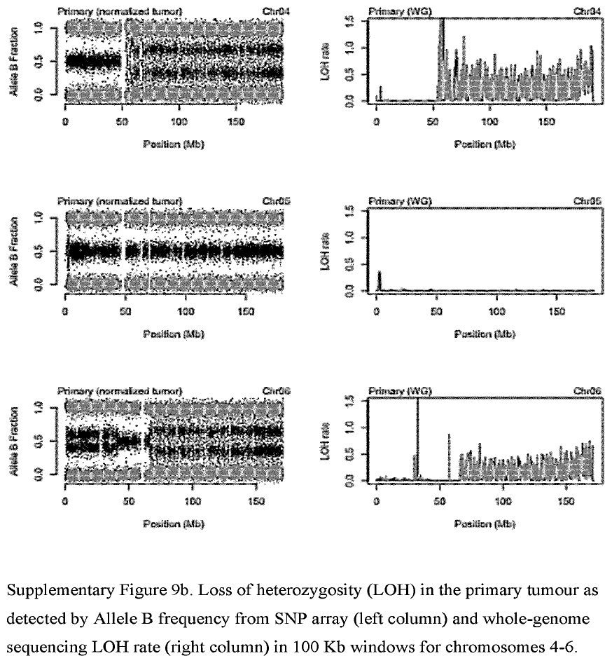

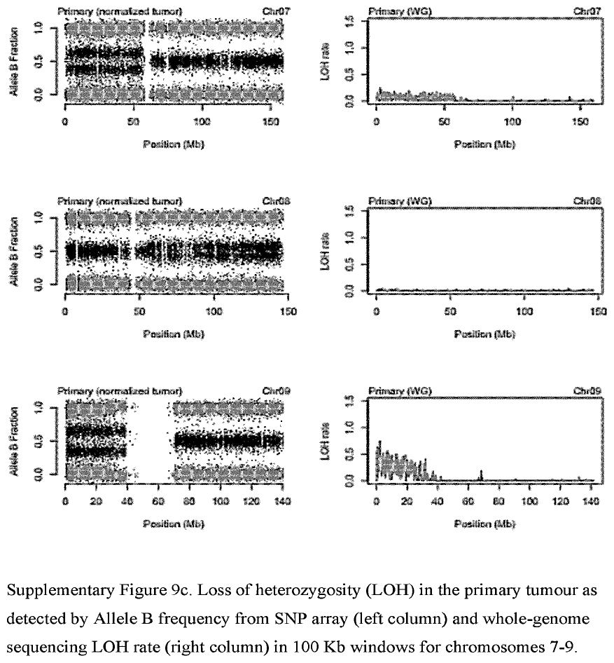

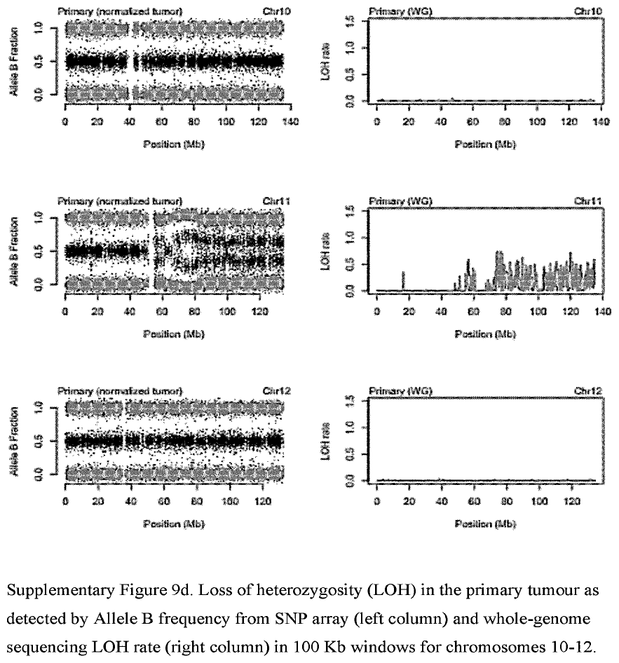

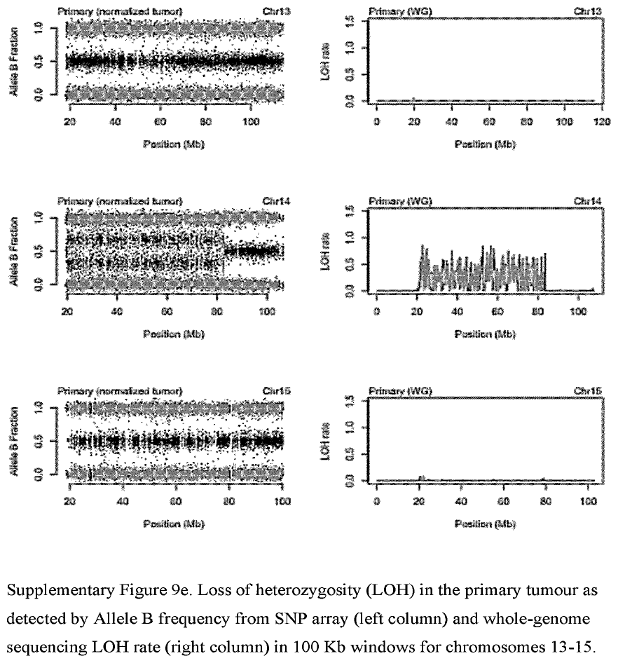

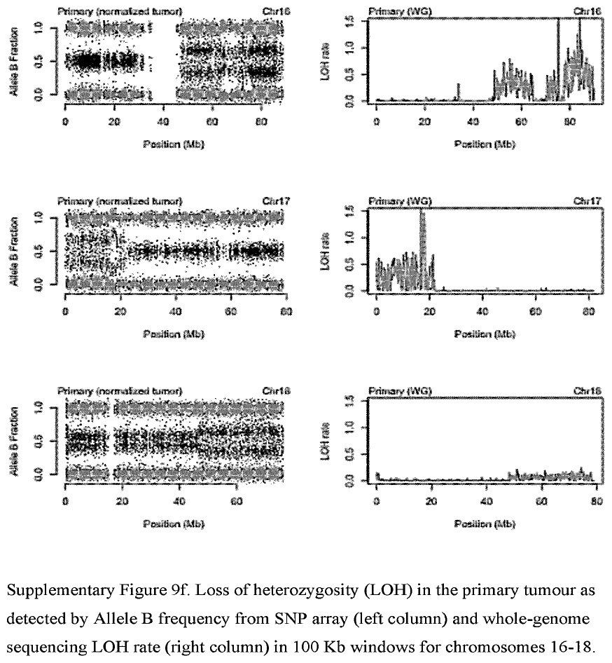

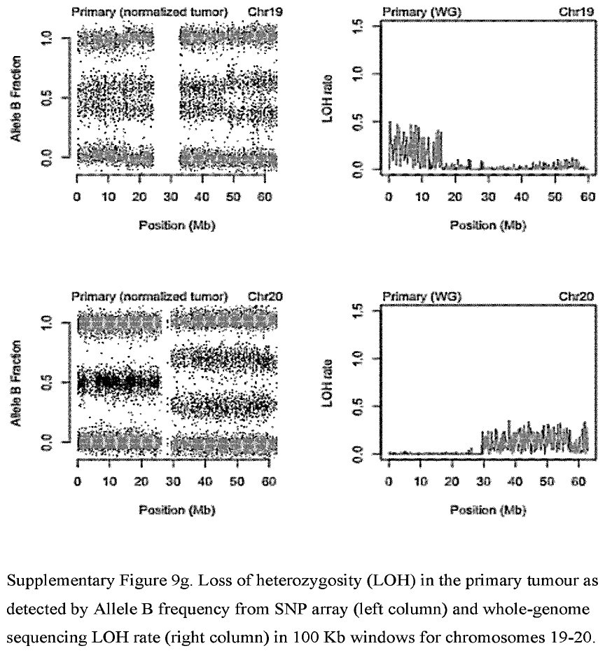

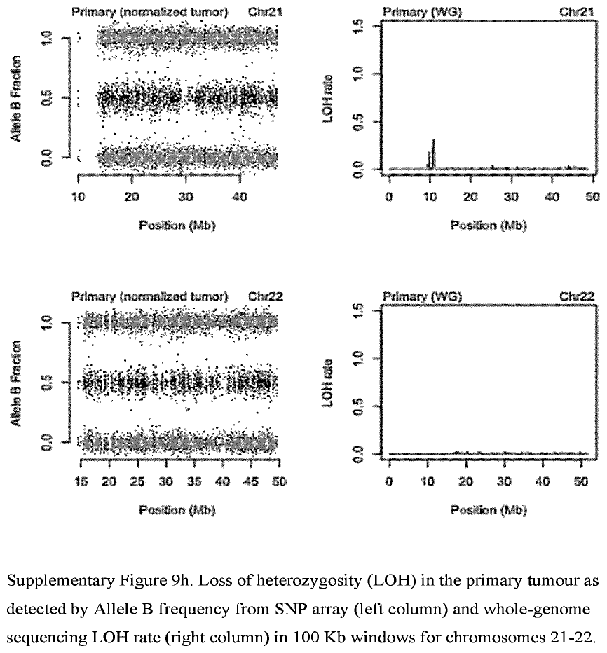

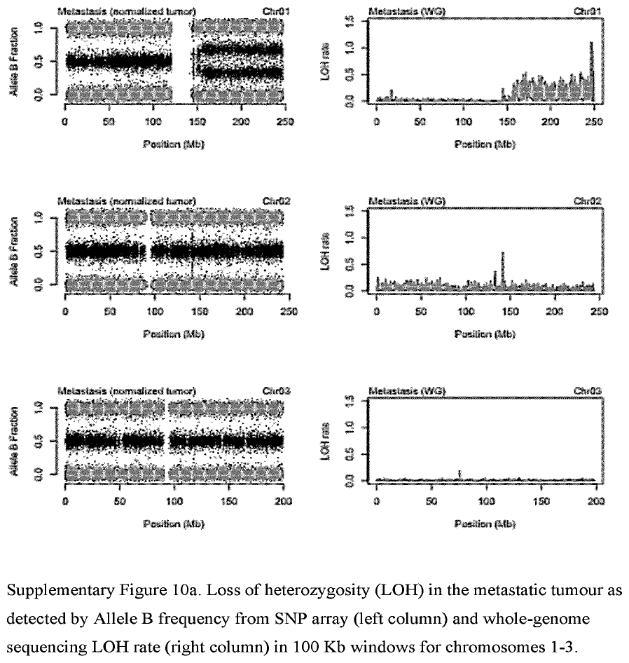

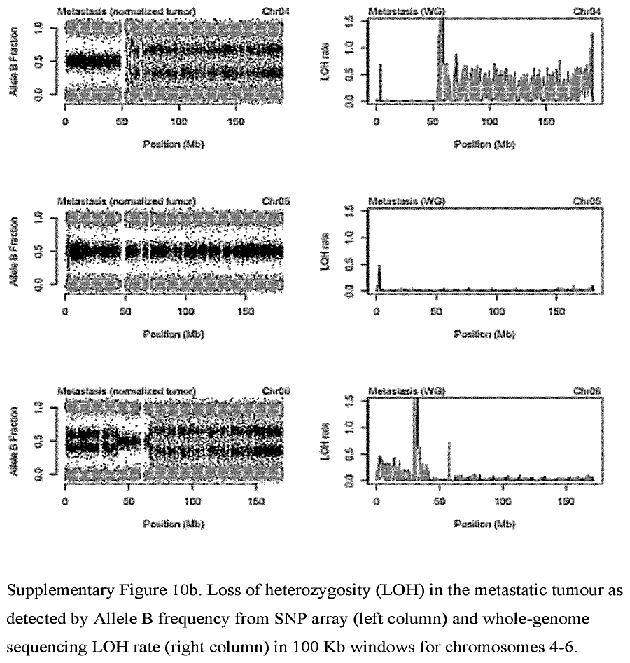

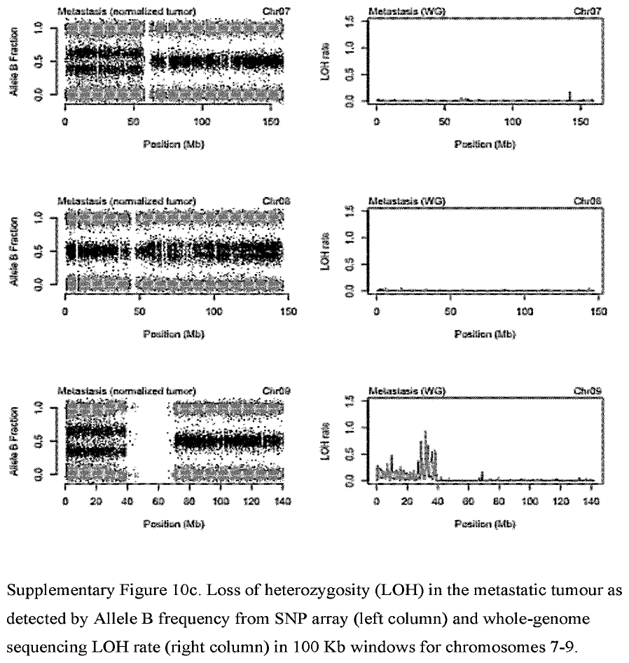

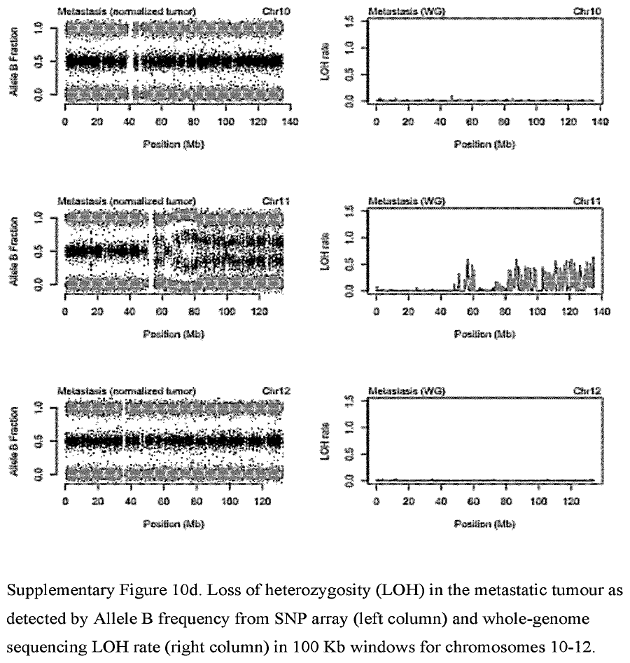

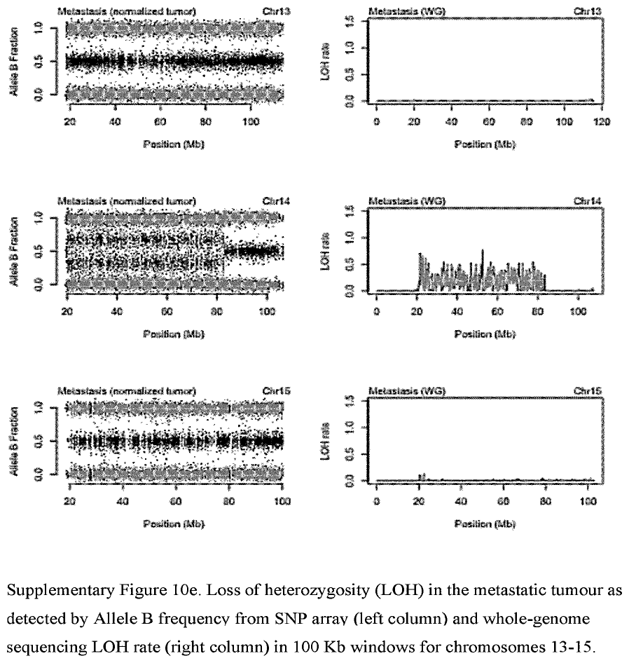

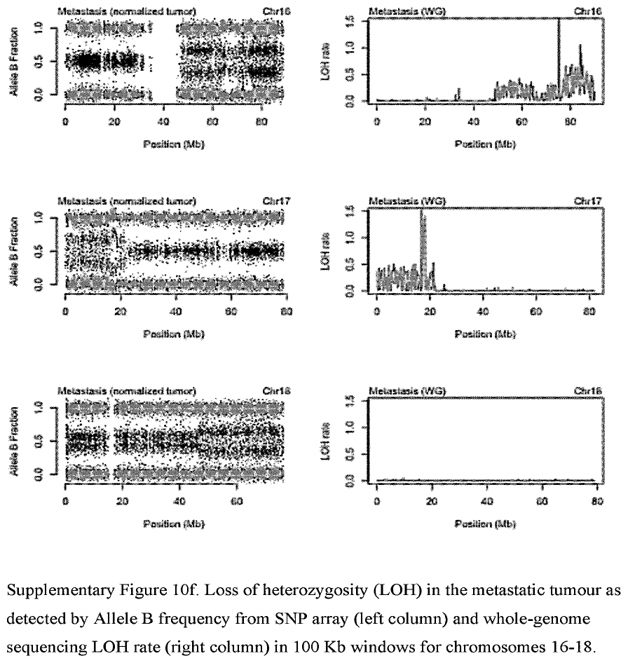

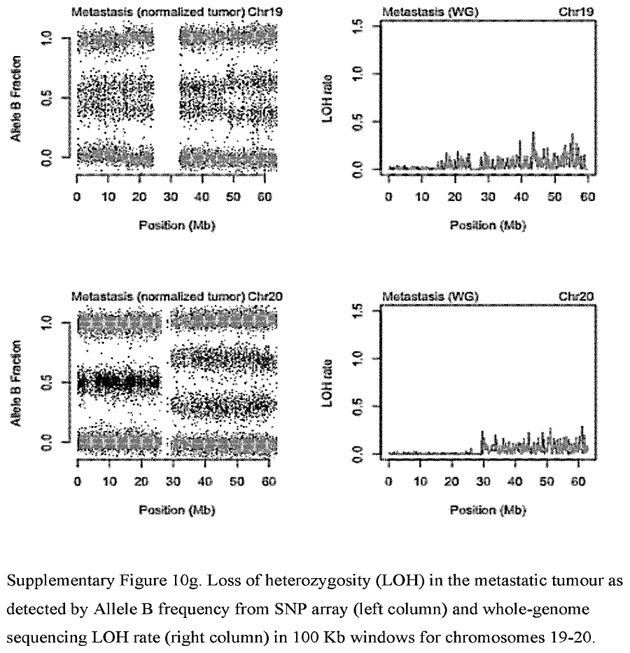

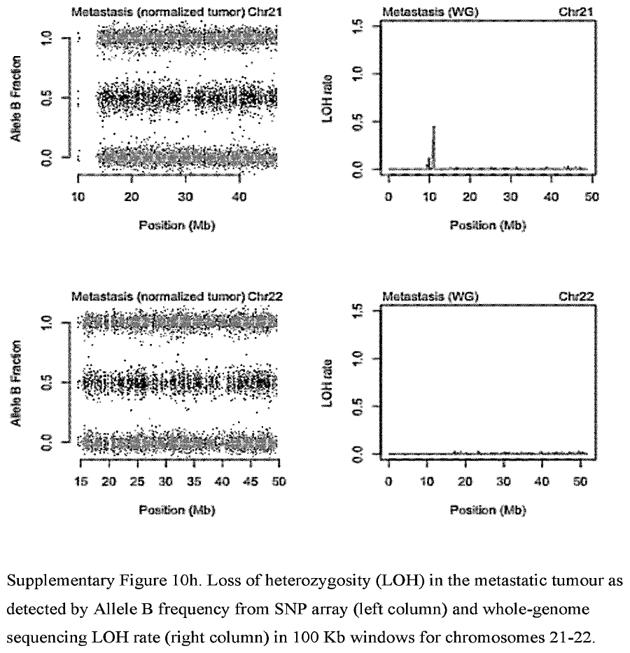

FIG. 9 shows a set of latent variables 900 generated by applying a signal-separation algorithm on sequence-size values corresponding to a biological sample of a subject diagnosed with colorectal cancer. A subset of latent variables 905 can be selected from the set of latent variables 900, in which the subset of latent variables 905 identify size distributions of sequences that are representative of the corresponding biological sample fragmentomic signatures. FIG. 10 shows a set of latent variables 1000 generated by applying a signal-separation algorithm on sequence-size values corresponding to a biological sample of another subject diagnosed with colorectal cancer. A subset of latent variables 1005 can be selected from the set of latent variables 1000, in which the subset of latent variables 1005 identify size distributions of sequences that are representative of the corresponding biological sample fragmentomic signatures.

D. Biological Processes that Affect the Latent Variables

In some instances, the shape of the latent variables can be used to predict enrichment of protein molecule sets (e.g., of mono-nucleosome, di-nucleosome, mono-chromatosome, di-chromatosome and transcription factor complexes) in cell-free DNA sequence-size data, and in so predict binding of said molecule sets at the corresponding cellular loci in vivo, based on utilization of latent variables as statistical objects representative of the independently regulated molecules governing chromatin epigenetic states. In some instances, the latent variables are also used to predict potentially novel nucleic acid and protein entities and their corresponding structures based on their fragment length patterns.

For example, enrichment of a latent variable known to be associated with chromatosomes in the sequence data can be used to predict binding of chromatosomes and enrichment of silenced heterochromatin states at the corresponding cellular loci. In another example, enrichment of a latent variable that is associated with cancer in the sequence data can be used to predict whether a particular allele came from cancer cells. In yet another example, enrichment of a latent variable that is associated with gene expression in the sequence data can be used to predict whether a particular allele contributing to the sequencing data is being expressed. Additionally, or alternatively, relative cell-free DNA abundance of shorter and longer size types within a molecule set can be predicted by comparing bimodal latent variable density peaks, and, inferring cell-free DNA degradation kinetics from the rates of change in latent variable space.

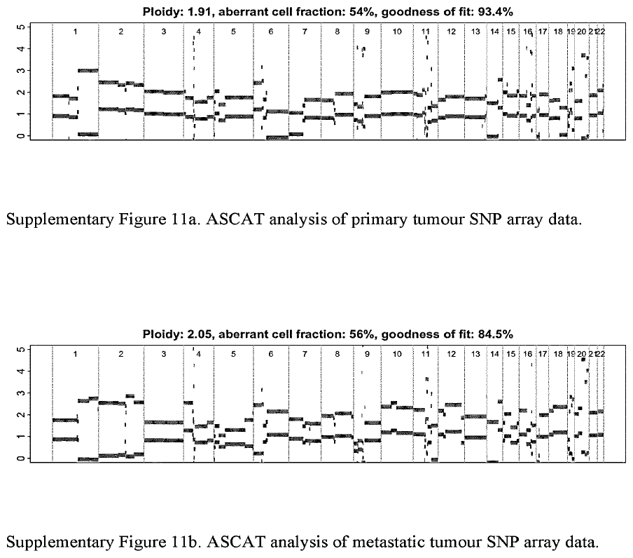

FIG. 11 shows a schematic diagram 1100 that illustrates a technique for predicting enrichment of protein molecule sets in sequence-size data, according to some embodiments.

A set of latent variables 1105 can include latent variables 1110a-e, each of which provides an estimated size distribution of nucleic-acid molecules in the biological sample. For each latent variable, a set of peaks can be identified. Each peak of the set of peaks can be used to predict the binding of a particular protein. For example, the latent variable 1110a can be represented as a unimodal size distribution with a peak 1115a at a sequence size of approximately 150 bp. The peak of such size distribution can be predictive of a location of a nucleosome binding when enriched in the sequence-size data associated with the genomic location. In addition to the peak 1115a, the periodic patterns of peaks having a peak-width of approximately 10 bp can be predictive of nucleosome binding by accounting for the effect of an additional DNA degradation signal, presumably correlated with progressive digestion of the discrete fragments associated with the nucleosome bound DNA helical pitch. The peaks can be correlated with behavior of nuclei acid molecules during apoptosis or necrotic cell death and the consequent shedding into and traversal of the circulatory system. For example, protein-bound DNA molecules, typically those associated with histones or transcription factors, preferentially survive damage (e.g., digestion) and are released into blood circulation, while unbound DNA molecules are lost.

Continuing with the examples in FIG. 11, some latent variables can be predictive of the binding of a particular protein. For example, the latent variable 1110e includes peaks 1120a-b represented together as a multimodal distribution, and each of the peaks 1120a-b can be used to predict transcription factor and transcription factor-dinucleosome complex binding when enriched in the sequence-size data associated with transcribed loci. In addition, the latent variable 1110e includes a third peak 1120c which has a larger peak-width value. Such peak can be used to predict transcription factor-mononucleosome binding. In addition to the widths of each of the peaks 1220a-b, the superimposed periodic patterns of approximately 10 bp presumably fingerprinting CTCF (e.g., as seen in ATAC-seq data) can be predictive of intercellular heterogeneity of DNA binding proteins governing transcribed locus epigenetic states.

The techniques described in FIG. 11 can be applied to a different set of latent variables 1125. The predicted protein binding can be used to define a nucleosome footprint pattern across the genome, which can then be used to predict whether a subject has cancer.

### E. Using Cancer-Derived Latent Variables for Calling Cancer-Related Gene Mutations

The latent variables of the fragmentomic signature can be used as features for predicting whether nucleic acid molecules of a biological sample carry cancer related mutations. For example, TP53 gene encodes a tumor suppressor protein which regulates cell division. It is one of the most frequently mutated genes in human cancer. TP53_chr17_7578265_A>C mutation was identified in CRC10063 cfDNA sample. The same mutation was also found in the paired tumor sample and not in the matched adjacent healthy tissue or white blood cell normal samples, indicating the mutation supporting reads from cfDNA are of tumor cell origin. The sequence-size distribution of these TP53 mutation supporting reads can be projected onto the fixed latent variables, resulting in a 1×5 vector represents the amplitude or enrichment of each latent variable in the sequence-size data. As a negative control, the same ICA projection was performed using high quality wildtype allele supporting reads. These two vectors can provide a way to quantify the fragment size difference.

FIG. 12 shows a schematic diagram 1200 that illustrates a process for pre-processing raw sequence-size distributions by projecting them onto latent variables, transforming the fragment size distribution into a set of amplitudes, and using the amplitudes to detect cancer-related gene mutations, according to some embodiments. As an initial step, BSS can be applied to sequence data obtained from cancer reference samples and repetitively identified latent variables across various reference samples are selected. At step 1205, after candidate mutations were called in cfDNA sample, mutation (ALT allele) supporting reads can be separated from wildtype allele (REF allele) supporting reads. At step 1210, the sequence-size distributions of the ALT allele and the REF allele can be transformed into their respective PMFs and projected onto the fixed latent variables. The resulting latent variable amplitudes can be used to measure the quality of mutation calls. For example, the amplitudes of latent variables corresponding to mutation supporting reads from tumor tissue should be different from amplitudes of latent variables corresponding to wildtype supporting reads, while the amplitudes of mutation reads from experimental noise or errors should be more similar to those of wildtype reads.

It can be shown that latent variables can be highly enriched in sequence-size distributions of circulating tumor DNA (ctDNA) in a plasma sample. For example, a latent variable includes a weight vector generated by applying the ICA algorithm to sequence-size data of the late-stage colorectal cancer subject. Plasma cfDNA sequence-size data associated with the TP53 mutation mentioned above that is known to come from the matched tumor biopsy sample shows a narrow peak around 140-150 bp range and a wider peak around 200-300 bp range, which is very similar to the latent variable “LV5”.

In order to develop a model for filtering candidate mutation calls, sequence-size distributions corresponding to 78 loci with high quality mutations were selected and projected onto the fixed latent variables (see step 1210). At the same time, corresponding high quality wildtype reads were projected using the same method. The LV1 and LV5 amplitudes were plotted for each locus. The LV amplitude plot 1215 shows that a set of results corresponding to REF allele are clustered together in a first area 1220, whereas a set of results corresponding to the ALT allele are mostly distant from the cluster. In some instances, one or more cutoffs are determined based on the plotted results shown 1215. For example, a cutoff 1225 is approximately 0.4. Any result that are beyond the cutoff 1225 can be considered as having a somatic variant specific to tumor DNA. As an illustrative example, a result 1230 identified from the LV amplitude plot 1215 identifies a size distribution of nucleic acid molecules with known tumor-related variants (TP53 gene variants). VL Predicting Cancer using Latent Variables of Fragmentomic Signatures

The latent variables represented by the fragmentomic signatures can also be used for the early detection of cancer in subjects as well as the monitoring of cancer recurrence after treatment. Cancer patients usually have different cfDNA fragment-size distributions and end-motif frequencies compared with healthy control subjects. The size-distribution data of a particular subject can be projected onto the set of latent variables of the fragmentomic signature to generate fragmentomic signature amplitudes, at which the fragmentomic signature amplitudes can be used (e.g., via machine learning) to predict whether the particular subject has a disease (e.g., cancer). For example, the fragmentomic signature amplitudes can be compared with the fragmentomic signature amplitudes of a healthy donor to predict whether the subject has an abnormality. Other types of prediction can be considered based on the type of latent variables and the types of training data used for machine learning models. If the training data includes reference samples of patients have a particular stage of a disease, the latent variable based model can be used to predict whether the particular subject has the particular stage of the disease (e.g., stage IV cancer). If the training data includes reference samples with a particular type of a disease, the machine learning model can be used to predict whether the subject has the particular type of the disease (e.g., colorectal cancer).

A. Generating Latent Variables from a Set of cfDNA Samples Enriched by Tumor-Informed Panels

cfDNA analysis is an emerging method to detect minimal residual disease or cancer recurrence after treatment. However, its sensitivity is limited by the low tumor signals in plasma. The following description shows an example where tumor-derived DNA can be enriched by personalized hybridization capture, at which the tumor-enriched samples can then be used to derive latent variables for detecting cancer in subjects.

FIG. 13 shows a schematic diagram that illustrates an example technique of using independent component analysis algorithm on hybridization capture samples to generate a set of latent variables, according to some embodiments. In FIG. 13, normal/tumor/plasma sample trios across various tumor types were used. Whole-genome sequencing was performed using normal and tumor samples to detect somatic mutations in tumors. At step 1305, −1800 somatic mutations with high allele frequency were used to design a personalized hybridization capture panel for each patient. The tumor signals from the plasma can then be enriched using this tumor-informed panel. For each panel design, the hybridization captures were also performed using one or two healthy plasma samples as the reference.

To generate the latent variables, the sequencing reads were mapped to the reference genome and kept all reads within 50-550 bp. At step 1310, the fragment size PMF around each target region was generated for each plasma sample. At step 1315, the 1800×501 matrix identifying the sequence-size distributions of fragments were then used as input into a BSS algorithm, such as ICA. In this example, ICA was performed for each plasma sample and 10 latent variables (independent components) were kept.

Once latent variables were collected from 119 healthy and 101 patient plasma samples, K-means clustering was then used to identify similar latent variables across different samples (step 1320). Clustering algorithms were ran using different parameters, and an 8-cluster model was chosen according to several clustering evaluation methods. A result 1325 shows the centroid of each cluster, which is used as the latent variables for downstream analysis (e.g., to predict cancer in other subjects).

### B. Detecting Cancer Occurrence or Relapse Using Latent Variable Amplitudes

The following description shows an example of using the latent variables for monitoring cancer recurrence after surgery. FIG. 14 shows a schematic diagram that illustrates example techniques for monitoring cancer relapse using hand-crafted features or latent variable features, according to some embodiments. Hybridization capture panel design was performed according to the somatic mutations of the tumor sample. At step 1405, plasma samples were collected at different time points after surgery, hybridization capture was then used to enrich the tumor sample for target cfDNA. As a reference, hybridization capture was also performed using a healthy plasma sample. At step 1410, in order to monitor possible cancer recurrence at different time points, the fragment size distributions from both patient and healthy samples can be extracted and projected onto the 8 fixed latent variables. At step 1415, a pre-trained machine learning model can then be used to compare patient latent variable amplitudes with control latent variable amplitudes, and assign status for different time points. At step 1420, to compare the latent-variable approach with other techniques, a baseline model was also trained using 13 hand-crafted features, such as the cumulative probability of reads within 50-250 bp, the probability of reads within 160-170 bp, and the ratio of 250-300 bp reads to 300-350 bp reads.

Regarding the machine-learning models, two logistic regression models based on hand-crafted (step 1420) or latent variable features (step 1415) were trained using a dataset of 43 healthy-healthy pairs and 59 healthy-patient pairs. FIG. 15 shows a set of receiver operating characteristic (ROC) curves 1500 that show accuracy levels of using fragmentomic signatures for classification of diseases. In FIG. 15, graphs 1505 and 1510 show the receiver operating characteristic curves that identify performance of the two models. The latent variable features based model has better performance (the graph 1510) than the hand-crafted features based model (the graph 1505): training area under the curve increased from 95.8% to 97% and the cross-validation area under the curve (mean) increased from 93.8% to 97%. Therefore, BSS algorithms can be used effectively to extract data-driven latent variables features without bias. In addition, the latent variables can be used to obtain better performance than hand-crafted features in cancer detection.

VII. Methods for Predicting a Classification of a Disease of a Subject Based on Fragmentomic Signatures Determined from End-Motif Frequencies

FIG. 16 includes a flowchart 1600 illustrating an example of a method of for determining a fragmentomic signature of a subject based on end-motif frequencies of nucleic acid molecules of a biological sample, according to some embodiments. Some of the operations described in flowchart 1600 may be performed by a computer system (e.g., a computer system 1700 of FIG. 17). Although flowchart 1600 may describe the operations as a sequential process, in various embodiments, many of the operations may be performed in parallel or concurrently. In addition, the order of the operations may be rearranged. An operation may have additional steps not shown in the figure. Furthermore, some embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

At step 1602, sequence data of a biological sample of a subject can be accessed. In some instances, the sequence data correspond to a plurality of cell-free DNA molecules of the biological sample, and the plurality of cell-free DNA molecules include circulating-tumor DNA molecules. The sequence data can also include sequences corresponding to a plurality of somatic variants detected from the biological sample. In addition, each cell-free DNA molecule can include a corresponding end motif. An end motif can be identified as an ending sequence of nucleotides of a DNA fragment, e.g., sequence for the K bases at either end of the fragment. The ending sequence can be a k-mer having various numbers of bases, e.g., 1, 2, 3, 4, 5, 6, 7. In some instances, the end motif is determined by aligning the sequence reads to a reference genome and identify nucleotide bases just before a start position or just after an end position. Such bases will still correspond to ends of the DNA fragments, e.g., as they are identified based on the ending sequences of the fragments. The process for identifying end motifs in cell-free DNA molecules are further described in Section III.B of the present disclosure.

At step 1604, based on the sequence data, a set of end-motif sequence data can be generated. In some instances, each end-motif sequence data of the set identifies a number or relative frequency of nucleic acid molecules having an ending sequence that correspond to a particular end-motif. For example, the set of end-motif sequence data can include, for a particular 4-mer end motif (e.g., CCCA), a count of sequence reads having the end motif. In some instances, the number or relative frequency of nucleic acid molecules having the particular end-motif can be normalized (e.g., using a total count of sequence reads in the biological sample).

At step 1606, the set of end-motif sequence data can be projected onto latent variables of a fragmentomic signature. The fragmentomic signature can include one or more signatures of distributions of end-motif frequencies of nucleic acid molecules that can be predictive of the classification of the disease. The fragmentomic signature can include one or more fixed latent variables. The fixed latent variables can be determined by: (i) applying one or more signal-separation algorithms to end-motif sequence data obtained from one or more reference samples to generate a set of latent variables; and (ii) applying a clustering algorithm to select a subset of the set of latent variables, in which the subset correspond to the fixed latent variables. In some instances, the reference samples include biological samples (e.g., tissue, plasma sample) obtained from subjects with disease diagnoses (e.g., cancer). Additionally or alternatively, the reference samples can include a biological sample obtained from the same subject but at a different time point. In some instances, each latent variable of the set of latent variables includes a histogram or weight vector that represents a distribution of end-motif frequencies of the reference samples. The one or more signal-separation algorithms can be a blind-source separation algorithm that can include an independent component analysis algorithm and/or a non-negative matrix factorization algorithm.

Additionally or alternatively, derivatives of latent variables can be determined by applying transformations such as scaling, translating, averaging, frequency domain converting (e.g., fast Fourier transforming) and other similar transformations.

Plasma DNA nucleases such as DFFB, DNASE1L3, and DNASE1 are involved in both cfDNA generation and clearance. The cleavage preference of these different nucleases can affect cfDNA end-motif frequency. Studies show that the activity of plasma DNA nucleases can be modified by multiple diseases, such as cancer and lupus erythematosus. In some instances, given that the BSS algorithms have the record of estimating signals carried by physically separate entities, the set of latent variables can be predictive of the activity of plasma DNA nucleases in different subjects.

In addition, cfDNA end-motif frequency corresponds to the accessibility of DNA nucleases to different genomic regions. Therefore, latent variables can be predictive of the degree of intercellular heterogeneity of bound DNA and thus of the proteins enabling the epigenetic states.

At step 1608, one or more fragmentomic signature amplitudes of the biological sample can be determined based on projecting the set of end-motif sequence data onto the set of latent variables. The fragmentomic signature amplitudes can be determined by projecting the set of end-motif sequence data (e.g., PMFs) onto a subset of the set of latent variables of the reference samples. In some instances, a clustering algorithm is applied to the latent variables generated from different reference samples to determine the subset of the set of latent variables. The subset of latent variables can include the centroids or medoids selected from the identified clusters of latent variables.

At step 1610, the fragmentomic signatures amplitudes can be used as input to a machine-learning algorithm (e.g., a logistic-regression classifier model) to generate a result. The result can be predictive of a classification predictive of whether the subject has a particular disease. The particular disease can include cancer. In some instances, if the reference samples were obtained from the same subject but at different time points, the result is predictive of a progression or relapse of the particular disease. The result can be used to identify a treatment for the subject and/or determine how often the treatment should be administered to the subject. In another example, the result can be predictive of a presence of a chromatosome binding at, and silencing of, an allele of interest based on an algorithm determining significant enrichment of a mono- and di-chromatosome sequences associated with the allele.

Accordingly, by leveraging end-motif data generated from whole exome sequencing or certain genomic regions of the subject (for example), cfDNA mapped to each genomic region of the set of genomic regions can be modeled as a linear mixture of DNA molecules processed by different plasma DNA nucleases. The BSS algorithms can then be applied to the set of mixtures to estimate the fragmentomic signature of the subject.

At step 1614, the result can be outputted. For example, the result can be locally presented or transmitted to another device. The result can be outputted along with an identifier of the subject. Process 1600 terminates thereafter.

### VIII. Diseases and Treatments

Certain embodiments may include predicting, diagnosing, and/or prognosing a status or outcome of a disease or condition in a subject based on one or more biomedical outputs. Predicting, diagnosing, and/or prognosing a status or outcome of a disease in a subject may comprise diagnosing a disease or condition, predicting a disease or condition, predicting the stage of a disease or condition, assessing the risk of a disease or condition, assessing the risk of disease recurrence, assessing the efficacy of a drug, assessing risk of an adverse drug reaction, predicting optimal drug dosage, predicting drug resistance, or a combination thereof.

The samples disclosed herein may be from a pregnant woman. The sample can be maternal plasma samples which comprise fetal nucleic acid molecules. The fetus may carry chromosome aneuploidies. Fetal aneuploidies can cause various diseases, such as Down syndrome (trisomy 21), Patau syndrome (trisomy 13) and Edwards syndrome (trisomy 18). The fetus may carry diseases caused by gene mutations and deletions, such as Spinal Muscular Atrophy and DiGeorge syndrome.

The samples disclosed herein may be from a subject suffering from a cancer. The sample may comprise malignant tissue, benign tissue, liquid biopsy or a mixture thereof. The cancer may be a recurrent and/or refractory cancer. Examples of cancers include, but are not limited to, sarcomas, carcinomas, lymphomas or leukemias. In some instances, a sample comprising cancer tissue is obtained, but no matched normal sample is obtained. In some instances, no matched normal sample is available. In some instances, a matched normal sample is obtained (e.g., for training and testing of a model disclosed herein).

Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g., alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penile cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. The cancer may be a skin cancer, such as a basal cell carcinoma, squamous, melanoma, nonmelanoma, or actinic (solar) keratosis.

The cancer may be a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavitity (pleura) or lining of the abdomen (peritoneum). The mesothelioma may be due to asbestos exposure. The cancer may be a brain cancer, such as a glioblastoma.

The cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. The cancer may be a meningioma.

The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic leukemia.

Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenstrom macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

Certain embodiments may include treating and/or preventing a disease or condition in a subject based on one or more biomedical outputs. The one or more biomedical outputs may recommend one or more therapies. The one or more biomedical outputs may suggest, select, designate, recommend or otherwise determine a course of treatment and/or prevention of a disease or condition. The one or more biomedical outputs may recommend modifying or continuing one or more therapies. Modifying one or more therapies may comprise administering, initiating, reducing, increasing, and/or terminating one or more therapies. The one or more therapies comprise an anti-cancer, antiviral, antibacterial, antifungal, immunosuppressive therapy, or a combination thereof. The one or more therapies may treat, alleviate, or prevent one or more diseases or indications.

Examples of anti-cancer therapies include, but are not limited to, surgery, chemotherapy, radiation therapy, immunotherapy/biological therapy, photodynamic therapy. Anti-cancer therapies may comprise chemotherapeutics, monoclonal antibodies (e.g., rituximab, trastuzumab), cancer vaccines (e.g., therapeutic vaccines, prophylactic vaccines), gene therapy, or combination thereof.

### IX. Computing Environment

FIG. 17 illustrates an example of a computer system 1700 for implementing some of some embodiments disclosed herein. The computer system 1700 may include a distributed architecture, where some of the components (e.g., memory and processor) are part of an end user device and some other similar components (e.g., memory and processor) are part of a computer server. In some instances, the computer system 1700 is a computer system for determining fragmentomic signatures based on size distributions of nucleic acid molecules, which includes at least a processor 1702, a memory 1704, a storage device 1706, input/output (110) peripherals 1708, communication peripherals 1710, and an interface bus 1712. The interface bus 1712 is configured to communicate, transmit, and transfer data, controls, and commands among the various components of computer system 1700. The processor 1702 may include one or more processing units, such as CPUs, GPUs, TPUs, systolic arrays, or SIMD processors. Memory 1704 and storage device 1706 include computer-readable storage media, such as RAM, ROM, electrically erasable programmable read-only memory (EEPROM), hard drives, CD-ROMs, optical storage devices, magnetic storage devices, electronic non-volatile computer storage, for example, Flash® memory, and other tangible storage media. Any of such computer-readable storage media can be configured to store instructions or program codes embodying aspects of the disclosure. Memory 1704 and storage device 1706 also include computer-readable signal media.

A computer-readable signal medium includes a propagated data signal with computer-readable program code embodied therein. Such a propagated signal takes any of a variety of forms including, but not limited to, electromagnetic, optical, or any combination thereof. A computer-readable signal medium includes any computer-readable medium that is not a computer-readable storage medium and that can communicate, propagate, or transport a program for use in connection with computer system 1700.

Further, the memory 1704 includes an operating system, programs, and applications. The processor 1702 is configured to execute the stored instructions and includes, for example, a logical processing unit, a microprocessor, a digital signal processor, and other processors. For example, the computing system 1700 can execute instructions (e.g., program code) that configure the processor 1702 to perform one or more of the operations described herein. The program code includes, for example, code implementing the analyzing the sequence data, and/or any other suitable applications that perform one or more operations described herein. The instructions could include processor-specific instructions generated by a compiler or an interpreter from code written in any suitable computer-programming language, including, for example, C, C++, C#, Visual Basic, Java, Python, Perl, JavaScript, R and ActionScript.

The program code can be stored in the memory 1704 or any suitable computer-readable medium and can be executed by the processor 1702 or any other suitable processor. In some embodiments, all modules in the computer system for performing the various features and processes described herein are stored in the memory 1704. In additional or alternative embodiments, one or more of these modules from the above computer system are stored in different memory devices of different computing systems.

The memory 1704 and/or the processor 1702 can be virtualized and can be hosted within another computing system of, for example, a cloud network or a data center. I/O peripherals 1708 include user interfaces, such as a keyboard, screen (e.g., a touch screen), microphone, speaker, other input/output devices, and computing components, such as graphical processing units, serial ports, parallel ports, universal serial buses, and other input/output peripherals. The I/O peripherals 1708 are connected to the processor 1702 through any of the ports coupled to the interface bus 1712. The communication peripherals 1710 are configured to facilitate communication between the computer system 1700 and other computing devices over a communications network and include, for example, a network interface controller, modem, wireless and wired interface cards, antenna, and other communication peripherals. For example, the computing system 1700 is able to communicate with one or more other computing devices (e.g., a computing device that determines fragmentomic signatures based on size distributions of nucleic acid molecules, another computing device that generates sequence data of a biological sample of the subject) via a data network using the a network interface device of the communication peripherals 1710.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art. Indeed, the methods and systems described herein may be embodied in a variety of other forms; furthermore, various omissions, substitutions and changes in the form of the methods and systems described herein may be made without departing from the spirit of the present disclosure. The accompanying claims and their equivalents are intended to cover such forms or modifications as would fall within the scope and spirit of the present disclosure.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provide a result conditioned on one or more inputs. Suitable computing devices include multipurpose microprocessor-based computing systems accessing stored software that programs or configures the computing system from a general purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Certain embodiments of the methods disclosed herein may be performed in the operation of such computing devices. The order of the blocks presented in the examples above can be varied—for example, blocks can be re-ordered, combined, and/or broken into sub-blocks. Certain blocks or processes can be performed in parallel.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another, or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

1. A method comprising:
(a) accessing sequence data of a biological sample of a subject;
(b) generating, based on the sequence data, a first set of sequence-size values, wherein each sequence-size value of the first set corresponds to a size of a sequence of the sequence data;
(c) determining fragmentomic signature amplitudes of the subject by projecting the first set of sequence-size values onto latent variables of a fragmentomic signature, wherein the latent variables are generated by applying one or more signal-separation algorithms to other set(s) of sequence-size values obtained from one or more reference biological samples;
(d) generating a result by processing the fragmentomic signature amplitudes using a machine-learning model, wherein the result includes a classification predictive of whether the subject has a particular disease; and
(e) outputting the result.

2. The method of claim 1, wherein each latent variable of the latent variables of the fragmentomic signature includes a histogram or a weight vector that represents a size distribution of the other set(s) of sequence-size values of the one or more reference biological samples, and wherein the fragmentomic signature amplitudes of the biological sample are determined by projecting the first set of sequence-size values and other set(s) of sequence-size values onto each latent variable of the latent variables.

3. The method of claim 1, wherein the first set of sequence-size values correspond to sequences of the sequence data that align to one or more genomic regions.

4. The method of claim 1, wherein the one or more signal-separation algorithms include one or more blind-source separation algorithms.

5. The method of claim 4, wherein the one or more blind-source separation algorithms further include an independent component analysis algorithm.

6. The method of claim 4, wherein the one or more blind-source separation algorithms further include a non-negative matrix factorization algorithm.

7. The method of claim 1, wherein one or more graph components of a first latent variable of the latent variables are predictive of a progressive digestion of DNA fragments associated with nucleosome-bound DNA helical pitch.

8. The method of claim 1, further comprising wherein one or more graph components of a second latent variable of the latent variables are predictive of intercellular heterogeneity of DNA binding proteins.

9. The method of claim 1, wherein each sequence represented by a corresponding sequence-size value includes a DNA fragment having a size ranging between 60 bp and 600 bp.

10. The method of claim 1, wherein the sequence data includes sequences corresponding to a plurality of somatic variants detected from the biological sample.

11. The method of claim 1, wherein the set of sequence-size values further is an empirical probability mass function generated based on the sequences of the sequence data.

12. The method of claim 1, wherein the sequence data corresponds to a plurality of cell-free DNA molecules of the biological sample, and wherein the plurality of cell-free DNA molecules includes circulating-tumor DNA molecules.

13. The method of claim 1, wherein the particular disease is cancer.

14. The method of claim 1, wherein the determining fragmentomic signature amplitudes of the subject includes projecting the first set of sequence-size values onto each of a subset of the latent variables.

15. The method of claim 14, wherein the subset of latent variables of the fragmentomic signature includes applying a clustering algorithm to the latent variables.

16. The method of claim 14, wherein the subset of the latent variables of the fragmentomic signature are used as pre-processing training data for another machine-learning model.

17. The method of claim 14, wherein the subset of the latent variables of the fragmentomic signature are used as components of a subsequent principal component analysis.

18. The method of claim 1, further comprising:
(i) generating, based on the sequence data, a set of end-motif sequence data, wherein each end-motif sequence data of the set identifies a number or relative frequency of nucleic acid molecules having an ending sequence that correspond to a particular end-motif; and
(ii) determining latent variables of another fragmentomic signature by applying one or more signal-separation algorithms to the set of end-motif sequence data.

19. The method of claim 18, wherein the determining the fragmentomic signature amplitudes of the subject includes projecting the set of end-motif sequence data onto the latent variables of the other fragmentomic signature.

20. A system comprising:
one or more data processors; and
a non-transitory computer readable storage medium containing instructions which, when executed on the one or more data processors, cause the one or more data processors to perform;
(a) accessing sequence data of a biological sample of a subject;
(b) generating, based on the sequence data, a first set of sequence-size values, wherein each sequence-size value of the set corresponds to a size of a sequence of the sequence data;
(c) determining fragmentomic signature amplitudes of the subject by projecting the first set of sequence-size values onto latent variables of a fragmentomic signature, wherein the latent variables are generated by applying one or more signal-separation algorithms to other set(s) of sequence-size values obtained from one or more reference biological samples;
(d) generating a result by processing the fragmentomic signature amplitudes using a machine-learning model, wherein the result includes a classification predictive of whether the subject has a particular disease; and
(e) outputting the result.

21. A computer-program product tangibly embodied in a non-transitory machine-readable storage medium, including instructions configured to cause one or more data processors to perform:
(a) accessing sequence data of a biological sample of a subject;
(b) generating, based on the sequence data, a first set of sequence-size values, wherein each sequence-size value of the set corresponds to a size of a sequence of the sequence data;
(c) determining fragmentomic signature amplitudes of the subject by projecting the first set of sequence-size values onto latent variables of a fragmentomic signature, wherein the latent variables are generated by applying one or more signal-separation algorithms to other set(s) of sequence-size values obtained from one or more reference biological samples;
(d) generating a result by processing the fragmentomic signature amplitudes using a machine-learning model, wherein the result includes a classification predictive of whether the subject has a particular disease; and
(e) outputting the result.
